(19) United States
(12) Patent Application Publication
(10) Pub. No.: US 2024/0193772 A1
(43) Pub. Date: Jun. 13, 2024

(54) QUANTIFYING THE TUMOR-IMMUNE ECOSYSTEM IN NON-SMALL CELL LUNG CANCER (NSCLC) TO IDENTIFY CLINICAL BIOMARKERS OF THERAPY RESPONSE

(71) Applicant: H. LEE MOFFITT CANCER CENTER AND RESEARCH INSTITUTE, INC., Tampa, FL (US)

(72) Inventors: Alexander R. A. ANDERSON, Tampa, FL (US); Mark ROBERTSON-TESSI, Lutz, FL (US); Chandler D. GATENBEE, Tampa, FL (US); Sandhya PRABHAKARAN, Tampa, FL (US)

(21) Appl. No.: 18/556,405

(22) PCT Filed: Apr. 22, 2022
(86) PCT No.: PCT/US2022/025910

§ 371 Date: Oct. 20, 2023

Related U.S. Application Data
(60) Provisional application No. 63/178,126, filed on Apr. 22, 2021.
(60) Provisional application No. 63/188,607, filed on May 14, 2021.

(51) Int. Cl. G06T 7/00; G06T 5/70; G06T 7/11; G06T 7/136; G06V 10/82; G06V 20/69; G16H 20/40; G16H 30/40

(52) U.S. Cl. CPC G06T 7/0012; G06T 5/70; G06T 7/11; G06T 7/136; G06V 10/82; G06V 20/695; G06V 20/698; G16H 20/40; G16H 30/40; G06T 2207/20081; G06T 2207/20084; G06T 2207/30024; G06T 2207/30061; G06T 2207/30096; G06V 2201/03

(57) ABSTRACT

A method of processing medical image data to predict disease progression in non-small cell lung cancer (NSCLC) patients includes receiving a multiplexed tissue image comprising a plurality of cells stained for one or more markers, evaluating the multiplexed tissue image using a machine learning model, and predicting whether a patient's NSCLC will progress based on the evaluation of the multiplexed tissue image using the machine learning model.

### CROSS-REFERENCE TO RELATED APPLICATIONS

This application claims the benefit of and priority to U.S. Provisional Pat. App. No. 63/178,126, filed Apr. 22, 2021, and U.S. Provisional Pat. App. No. 63/188,607, filed May 14, 2021, each of which is incorporated herein in their entireties.

### STATEMENT REGARDING FEDERALLY FUNDED RESEARCH

This invention was made with government support under Grant No. U01CA232382 awarded by the National Cancer Institute. The government has certain rights in the invention.

### BACKGROUND

The present disclosure relates generally to a system and methods for predicting disease progression in non-small cell lung cancer patients.

Lung cancer is the leading cause of cancer death worldwide with 1.76 million people die as a result of the disease yearly. Non-small cell lung cancer (NSCLC) is the most prevalent type of lung cancer, accounting for about 85% of all cases. Disease progression and treatment response in NSCLC vary widely among patients. Therefore, accurate diagnosis is crucial in treatment selection and planning for each NSCLC patient. As targeted molecular therapies, immuno-oncology, combination therapies have become central in managing patients with NSCLC, the vital requirement for high throughput data analyses and clinical validation of biomarkers has become even more crucial. Multiplexed imaging of tissues, and their analysis, is an emerging and proficient approach aiding clinical cancer diagnosis and prognosis. Multiplexed images enable the precise interpretation of spatial distribution of cells and cellular states and the characterization of tumor-immune interactions in situ and at the single-cell level. Further, they allow simultaneous detection of various protein biomarkers on the same tissue sample permitting molecular and immune profiling of NSCLC, while preserving tumor tissue, and enable the prediction of response to a given treatment. However, image processing, and the subsequent interpretive and predictive tools for multiplexed image data, are severely lacking.

### SUMMARY

One implementation of the present disclosure is a method of processing medical image data to predict disease progression in non-small cell lung cancer (NSCLC) patients. The method includes receiving a multiplexed tissue image comprising a plurality of cells stained for one or more markers, evaluating the multiplexed tissue image using a machine learning model, and predicting whether a patient's NSCLC will progress based on the evaluation of the multiplexed tissue image using the machine learning model.

In some embodiments, the machine learning model is a supervised machine learning model.

In some embodiments, the supervised machine learning model is a classifier model and the classifier model classifies each of the plurality of cells as either stable or progressive.

In some embodiments, the classifier model is a support vector machine (SVM) classifier.

In some embodiments, the supervised machine learning model is a regressor model and the regressor model outputs a probability of NSCLC progression.

In some embodiments, the regressor model is a boosted regression tree (BRT).

Another implementation of the present disclosure is a method of processing medical image data to predict disease progression in non-small cell lung cancer (NSCLC) patients. The method includes receiving a multiplexed tissue image comprising a plurality of cells stained for one or more markers, evaluating the multiplexed tissue image using a machine learning classifier model by classifying each of the plurality of cells as either stable or progressive, and predicting whether a patient's NSCLC will progress based on the evaluation of the multiplexed tissue image using the machine learning classifier model.

In some embodiments, the method further includes preprocessing the multiplexed tissue image prior to evaluating the multiplexed tissue image by at least one of denoising the multiplexed tissue image using Otsu's method of automatic image thresholding, converting the multiplexed tissue image to grayscale, and tiling the multiplexed tissue image into a plurality of n pixel by m pixel frames, where n and m are integers greater than 0.

In some embodiments, evaluating the multiplexed tissue image further includes, prior to classifying the plurality of cells extracting cell segments from the multiplexed tissue image using a convolutional neural network, building a count matrix that compares the plurality of cells to the one or more markers from the extracted cell segments, clustering the count matrix to characterize cell type heterogeneity using a Gaussian mixture model, approximating tumor regions from the characterized cell types using multiple convex hulls, and identifying cellular neighborhoods based on the tumor regions.

In some embodiments, the multiplexed tissue image is a 7-stain image.

In some embodiments, the multiplexed tissue image is received from one of a medical imaging device or a database.

In some embodiments, the method further includes presenting an indication of the prediction to a user via a user interface.

In some embodiments, the method further includes generating a risk map that indicates a probability of NSCLC progression based on the prediction.

In some embodiments, the machine learning classifier model is a support vector machine (SVM).

In some embodiments, the method further includes parsing the multiplexed tissue image into a plurality of quadrants and evaluating each of the plurality of quadrants using a boosted regression tree (BRT), where the prediction of whether the patient's NSCLC will progress is further based on the output of the BRT and wherein the BRT outputs a probability of NSCLC progression for each of the plurality of quadrants.

Yet another implementation of the present disclosure is a method that includes processing medical image data to predict disease progression in non-small cell lung cancer (NSCLC) patients according to the method described above and administering treatment to the patient based on the prediction of whether the patient's NSCLC will progress.

In some embodiments, the step of administering treatment comprises starting, stopping, or altering an NSCLC treatment regimen.

Yet another implementation of the present disclosure is a method of processing medical image data to predict disease progression in non-small cell lung cancer (NSCLC) patients. The method includes receiving a multiplexed tissue image comprising a plurality of cells stained for one or more markers, parsing the multiplexed tissue image into a plurality of quadrants, and evaluating each of the plurality of quadrants using a machine learning regressor model, where the machine learning regressor model outputs a probability of NSCLC progression for each of the plurality of quadrants, and predicting whether a patient's NSCLC will progress based on the probability of NSCLC progression for each of the plurality of quadrants.

In some embodiments, the method further includes preprocessing the multiplexed tissue image prior to evaluating the multiplexed tissue image by at least one of denoising the multiplexed tissue image using Otsu's method of automatic image thresholding, converting the multiplexed tissue image to grayscale, and tiling the multiplexed tissue image into n pixel by m pixel frames, where n and m are integers greater than 0.

In some embodiments, evaluating the multiplexed tissue image further includes, prior to classifying the plurality of cells extracting cell segments from the multiplexed tissue image using a convolutional neural network, building a count matrix that compares the plurality of cells to the one or more markers from the extracted cell segments, clustering the count matrix to characterize cell type heterogeneity using a Gaussian mixture model, approximating tumor regions from the characterized cell types using multiple convex hulls, and identifying cellular neighborhoods based on the tumor regions.

In some embodiments, the multiplexed tissue image is a 7-stain image.

In some embodiments, the multiplexed tissue image is received from one of a medical imaging device or a database.

In some embodiments, the method further includes presenting an indication of the prediction to a user via a user interface.

In some embodiments, the method further includes generating a risk map that indicates a probability of NSCLC progression based on the prediction.

In some embodiments, the machine learning regressor model is a boosted regression tree (BRT).

In some embodiments, the method further includes evaluating the multiplexed tissue image using a support vector machine (SVM) classifier, wherein the SVM classifier classifies each of a plurality of cells shown in the multiplexed tissue image as either stable or progressive, and wherein the prediction of whether the patient's NSCLC will progress is further based on an output of the SVM classifier.

Yet another implementation of the present disclosure is a method including processing medical image data to predict disease progression in non-small cell lung cancer (NSCLC) patients according to the method described above and administering treatment to the patient based on the prediction of whether the patient's NSCLC will progress.

In some embodiments, wherein the step of administering treatment comprises starting, stopping, or altering an NSCLC treatment regimen.

Yet another implementation of the present disclosure is a system for processing medical image data related to non-small cell lung cancer (NSCLC). The system includes a processor and memory having instructions stored thereon that, when executed by the processor, cause the processor to perform operations including: receiving a multiplexed tissue image comprising a plurality of cells stained for one or more markers, evaluating the multiplexed tissue image using one of a support vector machine (SVM) classifier or a boosted regression tree (BRT), where the SVM classifier classifies each of the plurality of cells as either stable or progressive and where the BRT outputs a probability of NSCLC progression for each of the plurality of quadrants, and predicting whether a patient's NSCLC will progress based on the evaluation of the multiplexed tissue image using the SVM classifier or the BRT.

In some embodiments, the operations further include preprocessing the multiplexed tissue image prior to evaluating the multiplexed tissue image by at least one of denoising the multiplexed tissue image using Otsu's method of automatic image thresholding, converting the multiplexed tissue image to grayscale, and tiling the multiplexed tissue image into a plurality of n pixel by m pixel frames, where n and m are integers greater than 0.

In some embodiments, the operations further include presenting an indication of the prediction to a user via a user interface.

In some embodiments, the operations further include generating a risk map that indicates a probability of NSCLC progression based on the prediction. Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive, as claimed.

### BRIEF DESCRIPTION OF THE DRAWINGS

Various objects, aspects, features, and advantages of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

FIG. 1 is a block diagram of an image analysis system for predicting disease progression in NSCLC patients, according to some embodiments.

FIG. 2 is a graphical illustration of the two different computational approaches implemented by the system of FIG. 1, according to some embodiments.

FIGS. 3A-3G are illustrations of an image processing pipeline, according to some embodiments.

FIG. 4A is an illustration of quantifying tumor-immune cells at a tumor border using convex hull approximation, according to some embodiments.

FIG. 4B is a box plot showing the presence of Stromal T cells and T cells within tumors in both stable disease (SD) and progressive disease (PD) patients, according to some embodiments.

FIG. 4C is a box plot showing the number of immune and tumor cells in a tumor border in both SD and PD patients, according to some embodiments.

FIG. 5 is a graph showing the relatively proportions of tumor cells colocalized with one of more functional markers with respect to patient response category and treatment timing, according to some embodiments.

FIGS. 6A and 6B are t-distributed stochastic neighbor embedding (t-SNE) plots of three different clusters annotated with the differentially-expressed markers, according to some embodiments.

FIG. 6C is an example t-SNE plot generated by replacing the points of the t-SNE plots of FIGS. 6A and 6B with corresponding multiplexed images, according to some embodiments.

FIG. 7A is a cluster gram showing multiple distinction cellular neighborhoods (CNs) compared to their z-scored frequencies in each CN, according to some embodiments.

FIG. 7B is an example multiplexed field-of-view (FOV) with mapped CNs, according to some embodiments.

FIG. 7C is a two-dimensional (2D) t-SNE plot rendering of various cells colored by CN and patient category, according to some embodiments.

FIG. 7D show multiple versions of the t-SNE of FIG. 7C that each depict an expression spread of a different marker, according to some embodiments.

FIG. 7E is a box plot of the density of different CNs based on patient category, according to some embodiments.

FIG. 8 shows various plots of marker co-expression covariance captured by CNs across patient categories and corresponding t-SNE plots of marker expressions based on patient category and treatment timing, according to some embodiments.

FIGS. 9A and 9B are t-SNE plots of actual patient disease progression and predicted patient progression, according to some embodiments.

FIGS. 10A-10C are t-SNE plots of actual patient disease progression, predicted patient progression, and single-cell model accuracy, according to some embodiments.

FIGS. 11 and 12 are risk maps generated based on predicted disease progression, according to some embodiments.

FIG. 13A is a flow chart of a process for processing image data and training a predictive model, according to some embodiments.

FIG. 13B is a flow chart of a process for predicting disease progression using the predictive model of FIG. 13A, according to some embodiments.

FIG. 14A is a diagram of the spatial association between markers across patient categories, according to some embodiments.

FIG. 14B is a diagram of a progression probability prediction, according to some embodiments.

FIG. 14C shows multiple graphs of marker importance, according to some embodiments.

FIG. 14D is a graph of progression probability scores mapped onto quadrants of example SD and PD images, according to some embodiments.

FIG. 15 is a graph of marker abundance across all FOV quadrants, according to some embodiments.

FIG. 16 shows a risk map for an example PD patient, according to some embodiments.

FIG. 17A is a graph of the probably of disease progression per quadrant, according to some embodiments.

FIG. 17B is a graph of feature importance in predicting disease progression using the quadrant approach, according to some embodiments.

FIG. 17C includes variable response plots that indicate the risk of disease progression based on different markers, according to some embodiments.

FIG. 17D is a graph illustrating the interaction effects between variable markers, according to some embodiments.

FIGS. 18A-18D are t-SNE plots shown actual patient response and predicted progression scores, according to some embodiments.

FIG. 18E shows various plots of marker co-expression covariance captured by CNs across patient categories and corresponding t-SNE plots of marker expressions based on patient category and treatment timing, according to some embodiments.

FIG. 19 is an example t-SNE plot generated by replacing the points with corresponding multiplexed images, according to some embodiments.

FIG. 20A is a matrix of biological interaction between markers, according to some embodiments.

FIG. 20B shows network depictions of marker associations, according to some embodiments.

FIG. 21 is a flow chart of a process for predicting disease progression using a quadrant approach, according to some embodiments.

### DETAILED DESCRIPTION

Referring generally to the figures, a system and methods for predicting disease progression in NSCLC patients are shown, accordingly to various embodiments. More specifically, the system and methods described herein can predict whether a NSCLC patient's disease will progress or remain stable throughout treatment based on a machine learning based analysis of cellular image data. Cellular image data may be collected (i.e., provided to or received by) the system and may be used to train multiple predictive models to predict how a patient will respond to treatment (e.g., by remaining stable or progressing). As described herein, image data may refer to a multicolor Vectra images stained for CD3, PDL1, Pan-CK, PD1, CD8, DAPI, and FoxP3 (i.e., a 7-stain Vectra image). In particular, the image data may be a multiplexed image compiled from several individual images (e.g., one for each stain).

An in-depth analysis of multiplexed images based on the frequency, phenotype and spatial distribution of immune and tumor cells within the immune landscape of NSCLC has shown that distinct spatial cellular ecologies exist across progressive disease (PD) and stable disease (SD) patients, where tumors of PD patients are characterized by a highly suppressed immune environment prior to treatment enabling higher chances of disease progression during treatment. These fundamentally distinct architectures across PD and SD patients enable disease progression prediction and clinical biomarker identification. In development of the system and methods described herein, multiplexed images were obtained from nine patients with advanced/metastatic NSCLC, with progression, who were treated with an oral HDAC inhibitor (e.g., vorinostat) combined with a PD-1 inhibitor (e.g., pembrolizumab). Images were collected from all patients both pre-treatment and on-treatment and used to implement a computational multiplexed-image analysis pipeline using cell-segments and quadrats to analyze the spatial and temporal features of multiplexed NSCLC images.

The term “tumor” is defined herein as an abnormal mass of hyperproliferative or neoplastic cells from a tissue other than blood, bone marrow, or the lymphatic system, which may be benign or cancerous. In general, the tumors described herein are cancerous. As used herein, the terms “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of solid cancerous growths, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair. Examples of solid tumors are sarcomas, carcinomas, and lymphomas. Leukemias (cancers of the blood) generally do not form solid tumors.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Examples include, but are not limited to, lung carcinoma, adrenal carcinoma, rectal carcinoma, colon carcinoma, esophageal carcinoma, prostate carcinoma, pancreatic carcinoma, head and neck carcinoma, or melanoma. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

“Administration” of “administering” to a subject includes any route of introducing or delivering to a subject an agent. Administration can be carried out by any suitable means for delivering the agent. Administration includes self-administration and the administration by another.

The term “subject” or “patient” is defined herein to include animals such as mammals, including, but not limited to, primates (e.g., humans), cows, sheep, goats, horses, dogs, cats, rabbits, rats, mice, and the like. In some embodiments, the subject or patient is a human.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

The term “therapeutically effective” refers to the amount of the composition used is of sufficient quantity to ameliorate one or more causes or symptoms of a disease or disorder. Such amelioration only requires a reduction or alteration, not necessarily elimination.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

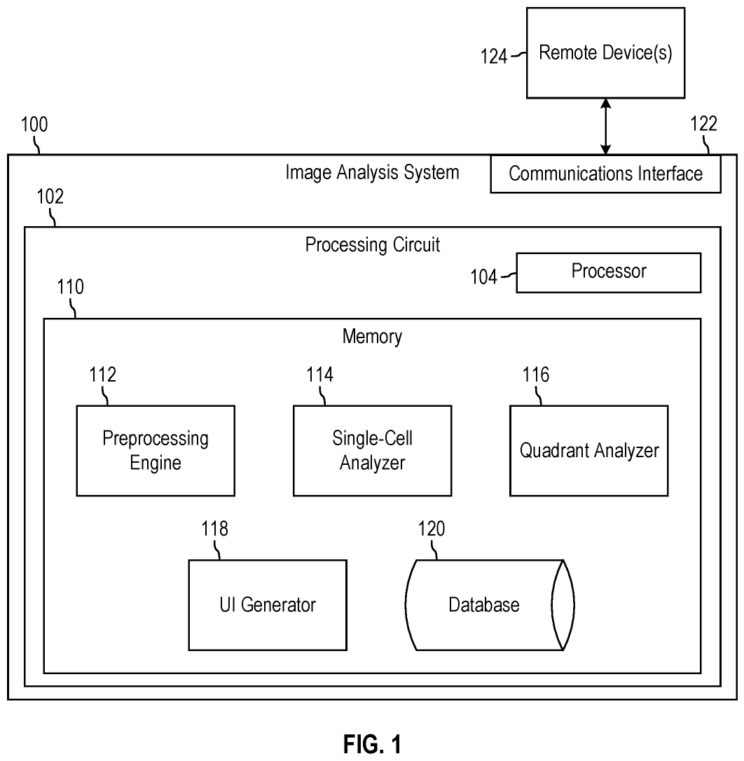

Turning first to FIG. 1, a block diagram of an image analysis system 100 for predicting disease progression in NSCLC patients is shown, according to some embodiments. At a high level, system 100 is configured to receive and process image data (e.g., a 7-stain multiplexed image stained for CD3, PDL1, Pan-CK, PD1, CD8, DAPI, and FoxP3) to generate a prediction on whether a given patient's NSCLC is expected to progress or remain stable through treatment. Multiplex immunochemistry (IHC) is an assay technique known in the art using single antigen staining to detect a plurality of biomarkers. In some implementations, the multiplexed tissue image is a 7-stain multiplexed image stained as described herein. It should be understood that the 7-stain multiplexed image stained as described herein is only provided as an example. This disclosure contemplates that the techniques described herein can be applied to other multiplexed image types. This prediction can subsequently be utilized to develop a treatment plan for the patient, for example. Advantageously, system 100 may implement two different image analysis techniques for highly accurate predictions.

System 100 is shown to include a processing circuit 102 that includes a processor 104 and a memory 110. Processor 104 can be a general-purpose processor, an application specific integrated circuit (ASIC), one or more field programmable gate arrays (FPGAs), a group of processing components, or other suitable electronic processing components. In some embodiments, processor 104 is configured to execute program code stored on memory 110 to cause system 100 to perform one or more operations. Memory 110 can include one or more devices (e.g., memory units, memory devices, storage devices, etc.) for storing data and/or computer code for completing and/or facilitating the various processes described in the present disclosure. In some embodiments, memory 110 includes tangible, computer-readable media that stores code or instructions executable by processor 104. Tangible, computer-readable media refers to any media that is capable of providing data that causes system 100 to operate in a particular fashion.

Example tangible, computer-readable media may include, but is not limited to, volatile media, non-volatile media, removable media and non-removable media implemented in any method or technology for storage of information such as computer readable instructions, data structures, program modules or other data. Accordingly, memory 110 can include random access memory (RAM), read-only memory (ROM), hard drive storage, temporary storage, non-volatile memory, flash memory, optical memory, or any other suitable memory for storing software objects and/or computer instructions. Memory 110 can include database components, object code components, script components, or any other type of information structure for supporting the various activities and information structures described in the present disclosure. Memory 110 can be communicably connected to processor 104, such as via processing circuit 102, and can include computer code for executing (e.g., by processor 104) one or more processes described herein.

While shown as individual components, it will be appreciated that processor 104 and/or memory 110 can be implemented using a variety of different types and quantities of processors and memory. For example, processor 104 may represent a single processing device or multiple processing devices. Similarly, memory 110 may represent a single memory device or multiple memory devices. Additionally, in some embodiments, system 100 may be implemented within a single computing device (e.g., one server, one housing, etc.). In other embodiments, system 100 may be distributed across multiple servers or computers (e.g., that can exist in distributed locations). For example, system 100 may include multiple distributed computing devices (e.g., multiple processors and/or memory devices) in communication with each other that collaborate to perform operations. For example, but not by way of limitation, an application may be partitioned in such a way as to permit concurrent and/or parallel processing of the instructions of the application. Alternatively, the data processed by the application may be partitioned in such a way as to permit concurrent and/or parallel processing of different portions of a data set by the two or more computers.

Memory 110 is shown to include a preprocessing engine 112 configured to receive and optionally preprocess image data. As mentioned above, image data may refer to a multicolor Vectra images stained for CD3, PDL1, Pan-CK, PD1, CD8, DAPI, and FoxP3 (i.e., a 7-stain Vectra image). In particular, the image data may be a multiplexed image compiled from several individual images (e.g., one for each stain). In some embodiments, image data is stored and/or received as a tag image file format (TIFF). Specifically, in some embodiments, a multiplexed image may be a TIFF stack (i.e., may contain multiple individual TIFF files). Each TIFF file, or sub-TIFF, may be associated with one type of marker, as mentioned above. In some embodiments, each sub-TIFF is an approximately 1008×1344 pixel image. In some embodiments, preprocessing engine 112 is configured to combine (i.e., aggregate) multiple images to form a multiplexed image. For example, preprocessing engine 112 may combine several individual images, each corresponding to one type of marker, to form a multiplexed image. In other embodiments, preprocessing engine 112 receives multiplexed images.

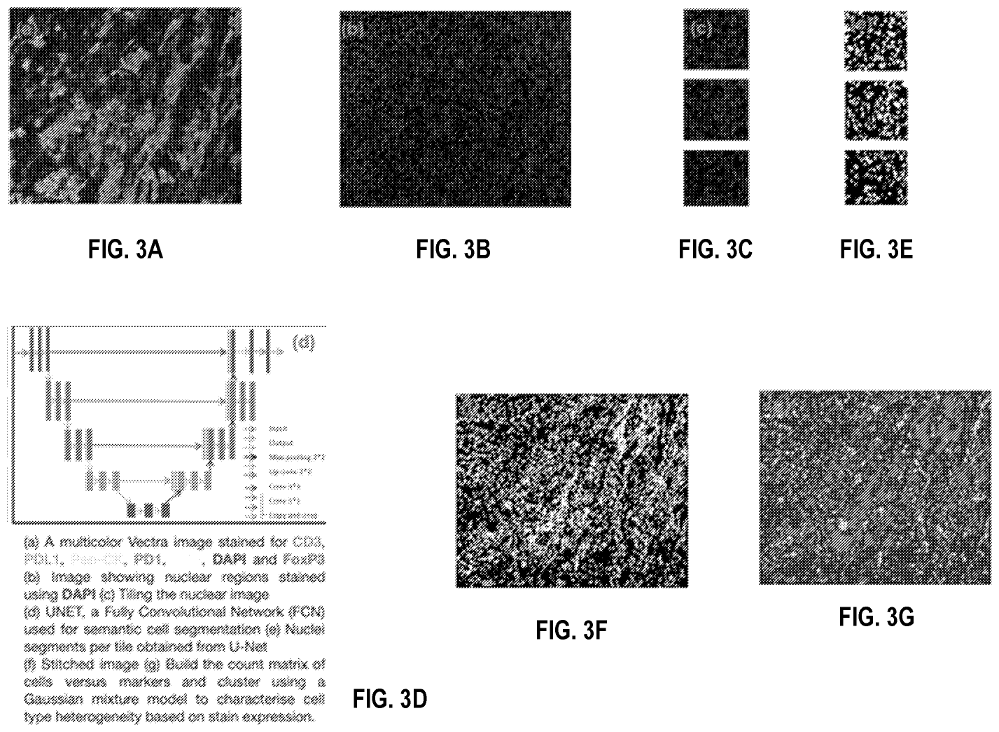

Generally, preprocessing of multiplexed images can include a number of different steps that may vary based on the type or size of image, the type of analysis to be performed, etc. Accordingly, all suitable preprocessing techniques are contemplated herein. As an example, preprocessing engine 112 may be configured to denoise (i.e., clean) each sub-TIFF of a TIFF stack (i.e., a multiplexed image). To denoise image data, preprocessing engine may implement Otsu's method of automatic image thresholding which involves iterating through all possible threshold values and calculating a measure of spread for the pixel levels each side of the threshold (e.g., the pixels that either fall in foreground or background). Foreground pixels may be regarded as true stain signals while background pixels may be interpreted as noise. Thus, background pixels can be filtered out or ignored. In some embodiments, preprocessing also includes generating a grayscale version of each sub-TIFF. In some embodiments, preprocessing engine 112 also tiles each sub-TIFF into smaller frames (e.g., 256×256 pixel frames). Additional description of various preprocessing techniques provided below with respect to FIG. 3.

In some embodiments, preprocessing engine 112 retrieves image data from a database 120. Database 120 may generally be configured to store and maintain image data, both pre- and post-processing. In some embodiments, database 120 stores a combination of sub-TIFF files (i.e., individual images related to a single marker), multiplexed images, and preprocessed images. In some embodiments, preprocessing engine 112 receives image data from one or more remote device(s) 124. In some such embodiments, preprocessing engine 112 stores the received image data in database 120 for later retrieval and/or preprocessing. In some embodiments, preprocessing engine 112 preprocesses the image data before storing the image data in database 120. In either case, image data may be received from remote device via a communications interface 122.

Communications interface 122 may facilitate communications between system 100 and any external components or devices (e.g., remote device(s) 124). For example, communications interface 122 can provide means for transmitting data to, or receiving data from, remote device(s) 124. Accordingly, communications interface 122 can be or can include a wired or wireless communications interface (e.g., jacks, antennas, transmitters, receivers, transceivers, wire terminals, etc.) for conducting data communications. In various embodiments, communications via communications interface 122 may be direct (e.g., local wired or wireless communications) or via a network (e.g., a WAN, the Internet, a cellular network, etc.). For example, communications interface 122 can include a WiFi transceiver for communicating via a wireless communications network. In another example, communications interface 122 may include cellular or mobile phone communications transceivers. In yet another example, communications interface 122 may include a low-power or short-range wireless transceiver (e.g., Bluetooth©).

As described herein, remote device(s) 124 may be any computing device(s) capable of sending and receiving image data. For example, remote device(s) 124 can include medical imaging devices, remote servers or computers, or the like. In general, remote device(s) 124 include a memory (e.g., RAM, ROM, Flash memory, hard disk storage, etc.) and a processor (e.g., a general-purpose processor, an application specific integrated circuit (ASIC), one or more field programmable gate arrays (FPGAs), a group of processing components, or other suitable electronic processing components). In some embodiments, remote device(s) 124 include a user interface (e.g., a touch screen), allowing a user to interact with system 100. Remote device(s) 124 can include, for example, mobile phones, electronic tablets, laptops, desktop computers, workstations, vehicle dashboards, and other types of electronic devices, in addition to medical imaging devices as mentioned above.

In some embodiments, memory 110 includes a user interface (UI) generator 118 for generating various graphical user interfaces (GUIs), some of which may be displayed via the user interface(s) of remote device(s) 124. For example, UI generator 118 may generate any of the graphics/images shown generally in the figures and described herein. In other words, UI generator 118 may be configured to generate GUIs including images, graphs, charts, text, etc. In some embodiments, these GUIs are displayed via a separate user interface (e.g., a display screen) of system 100 itself (not shown).

Still referring to FIG. 1, memory 110 is also shown to include a single-cell analyzer 114. At a high level, single-cell analyzer 114 is configured to receive image data, which may be or may include preprocessed image data from preprocessing engine 112 and/or database 120, and to generate a prediction of whether the NSCLC of a patient associated with the image data (i.e., a patient from which the image data was collected) will progress during treatment. In some embodiments, single-cell analyzer 114 executes a predictive model using the image data to generate said prediction. In some such embodiments, the predictive model is a binary support vector machine (SVM) which classifies data into one of two classes: “progression” or “non-progression/stable.” Additional description of the predictive model implemented by single-cell analyzer 114 is provided below. In some embodiments, single-cell analyzer 114 is also configured to train the predictive model using historical data. In general, the historical data may include a variety of image data and known outcomes (e.g., whether the patient's NSCLC progressed or remained stable).

Memory 110 is also shown to include a quadrant analyzer 116. At a high level, quadrant analyzer 116 is configured to receive image data, which may be or may include preprocessed image data from preprocessing engine 112 and/or database 120, and to generate a prediction of whether the NSCLC of a patient associated with the image data (i.e., a patient from which the image data was collected) will progress during treatment. Like single-cell analyzer 114, quadrant analyzer 116 executes a predictive model using the image data to generate said prediction. In some such embodiments, the predictive model is a boosted regression tree (BRT). Additional description of the predictive model implemented by quadrant analyzer 116 is provided below. In some embodiments, quadrant analyzer 116 is also configured to train the predictive model using historical data.

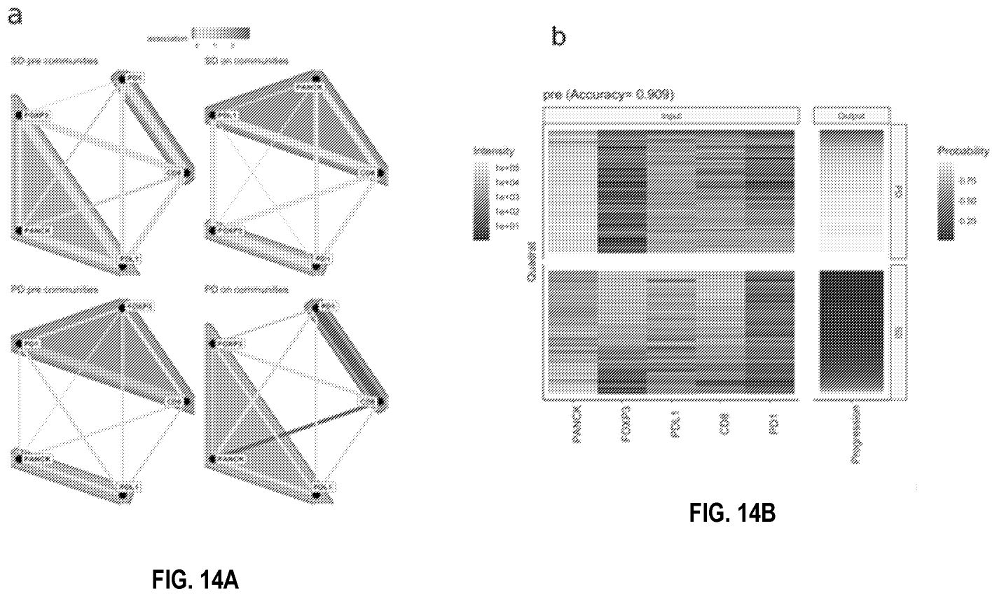

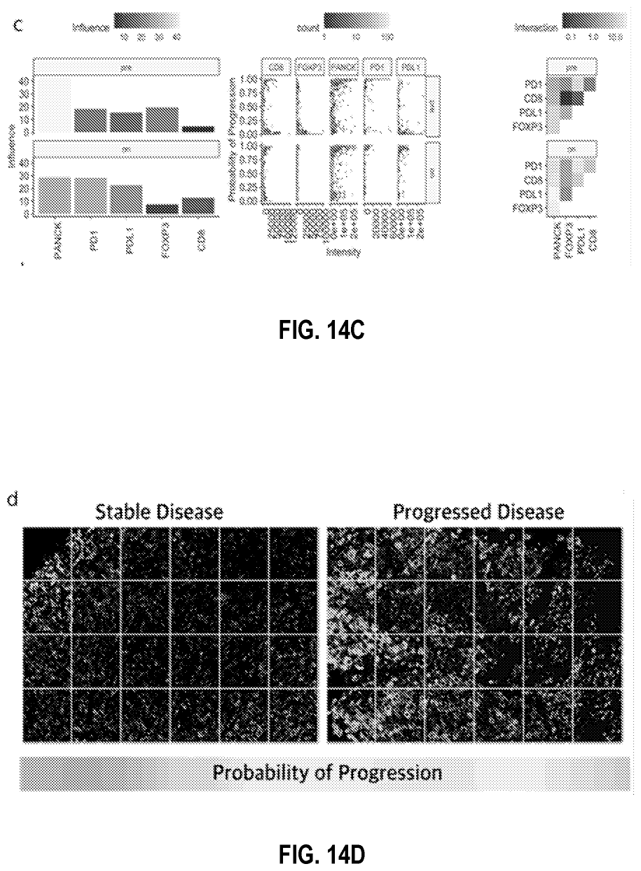

Additional features and advantages of system 100 are described in greater detail below. Specifically, it should be appreciated that the functions and advantages of single-cell analyzer 114 and quadrant analyzer 116 are only described above at a high-level for brevity; however, the functions and advantages of single-cell analyzer 114 and quadrant analyzer 116 will be made clearer with the description below. For example, single-cell analyzer 114 may implement the cell-segmentation image analysis technique described herein with respect to FIGS. 3-13B. Likewise, quadrant analyzer 116 may implement the quadrant-based image analysis technique described herein with respect to FIGS. 14A-21.

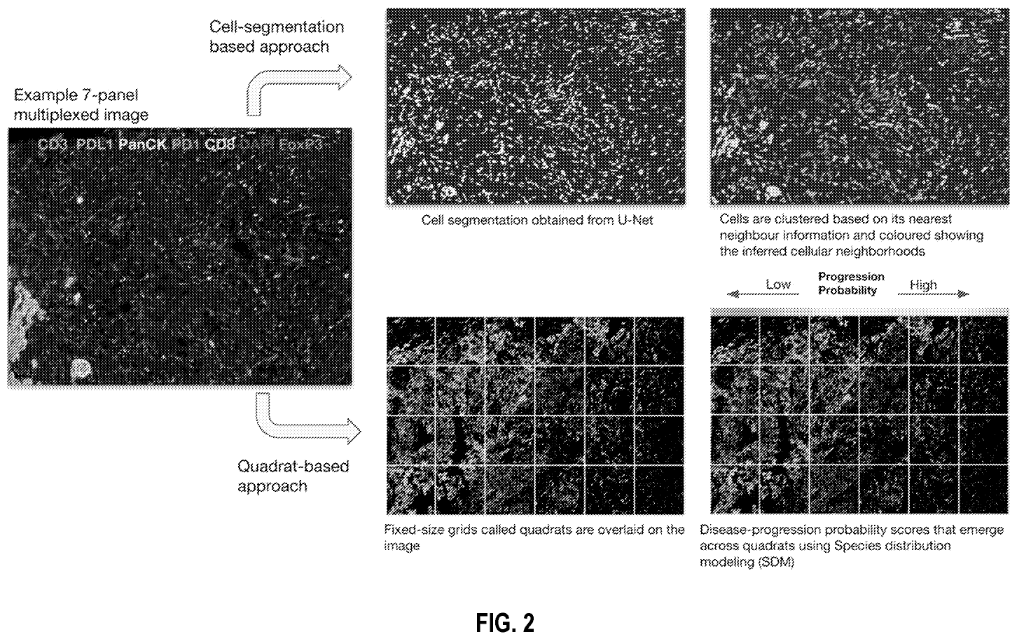

Referring now to FIG. 2, a graphical illustration of the two different computational approaches implemented by system 100 is shown, according to some embodiments. In particular, FIG. 2 provides an example of both a cell-segmentation based analysis of image data and a quadrant based analysis of image data. Both of these analysis techniques can be utilized to understand the spatial and temporal interactions between tumor and immune cells in NSCLC patients. In a cell-segmentation based analysis, as will be described in greater detail below, cell segments are extracted from image data (e.g., a multiplexed image) and used to build a count matrix having cells as rows and markers as columns. The count matrix may then be clustered to identify spatially heterogeneous cell types. Using a quadrant-based analysis, a multiplexed image is divided into a collection of fixed-size grids (i.e., quadrats) to study the distribution of cells and to detect global changes across the spatially diverse cell types.

### Cell-Segmentation Analysis

Referring now to FIGS. 3A-3G, an illustration of an image processing “pipeline” is shown, according to some embodiments. Specifically, FIGS. 3A-3G shows an example multiplexed image at various stages of processing. In some embodiments, the image processing steps show are implemented by one or both of preprocessing engine 112 and single-cell analyzer 114, described above. As shown, the example image is a 7-stain multiplexed Vectra image (FIG. 3A). As described above, the image data may be comprised of a plurality of TIFF files, including one TIFF for each marker. Accordingly, each TIFF may be individually processed as generally shown.

Initially, each pixel of the image (e.g., a 1008 pixel×1344 pixel sub-TIFF of the 7-stain Vectra TIFF stack) may be cleaned and/or denoised. Cleaning/denoising the image can include first generating a grayscale version of the image (i.e., the sub-TIFF) and choosing one marker for segmentation. In the example shown, a nuclear image stained with DAPI is chosen for cell segmentation (FIG. 3B). In some embodiments, automatic image thresholding (e.g., Otsu's method) is then applied to the image, which involves iterating through all possible threshold values and calculating a measure of spread for the pixel levels each side of the threshold (e.g., the pixels that either fall in foreground or background). Foreground pixels may be regarded as true stain signals while background pixels may be interpreted as noise. Thus, background pixels can be filtered out or ignored.

Subsequently, in some embodiments, the image is then tiled into smaller frames (e.g., 256×256 pixel frames) (FIG. 3C) and fed into a deep-learning architecture for segmentation. Specifically, the tiled image(s) may be fed into an artificial neural network. An artificial neural network (ANN) is a computing system including a plurality of interconnected neurons (e.g., also referred to as “nodes”) that can be trained to with a dataset to make predictions (e.g., classification or regression). This disclosure contemplates that the nodes can be implemented using a computing device (e.g., a processing unit and memory as described herein). ANNs are known in the art and therefore not described in further detail herein. The tiled image(s) described above may be fed into a convolutional neural network, such as U-Net (FIG. 3D), which predicts the cell segments (e.g., the nuclei of the cells) in the images (FIG. 3E). A convolutional neural network (CNN) is a type of deep neural network that has been applied, for example, to image analysis applications. CNNs are known in the art and therefore not described in further detail herein. The identified cell segments can then be stitched together (FIG. 3F). Finally, a count matrix with cells (rows) and markers (columns) is computed from the stitched, cell segmented image and clustering is performed to identify heterogeneous cell types (FIG. 3G). In some embodiments, cluster assignments are determined using a Gaussian mixture model which identifies heterogeneous cell types based on stain expression and, advantageously, does not require the number of clusters to be known in advance.

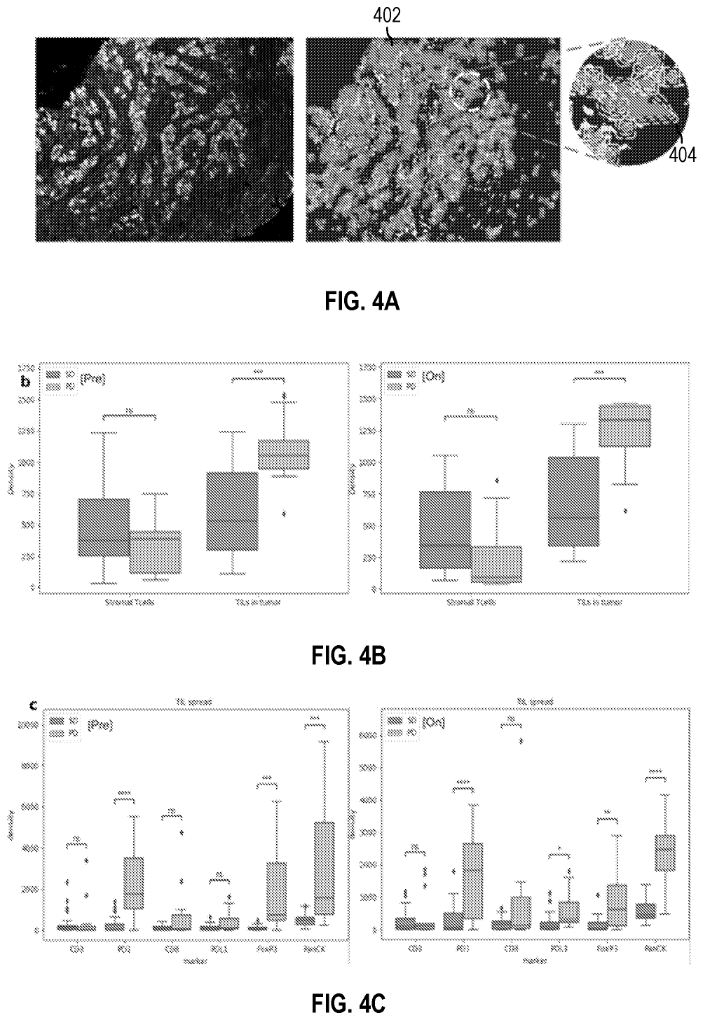

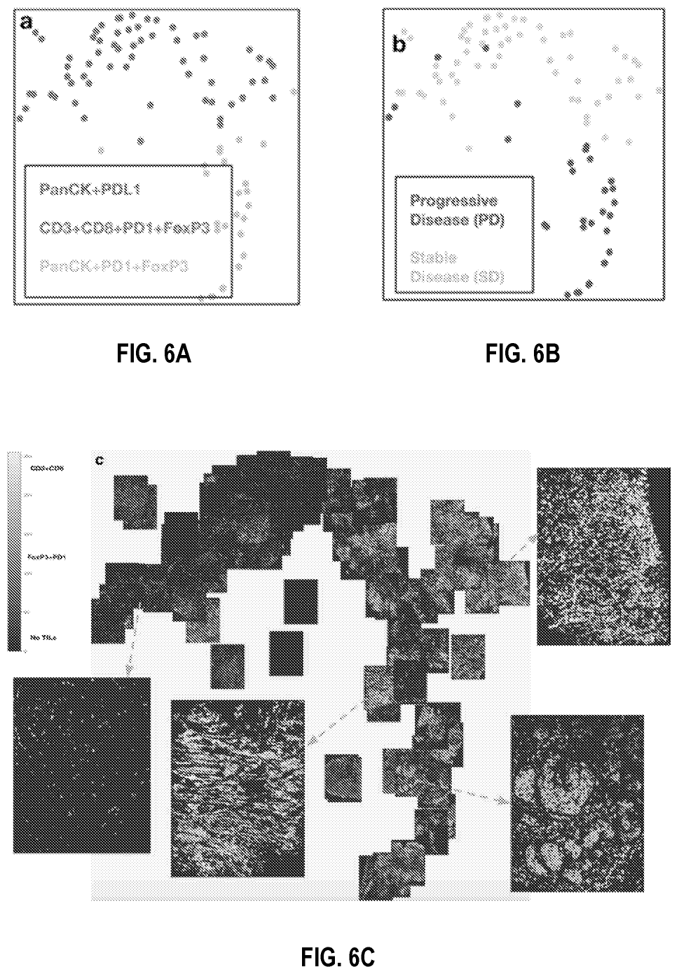

Based on the determined heterogeneous cell types (e.g., from clustering), tumor-rich regions can be identified. Specifically, single-cell analyzer 114 may automatically demarcate tumor-rich regions (e.g., across images) that are higher in PanCK expression, shown as regions 402 in FIG. 4A. Specifically, FIG. 4A shows an example 7-stain multiplex image and a corresponding copy of the original image having computationally identified tumor regions 402 and their boundary approximations using convex hulls 404. Multiple convex hulls 404 are shown extending into and outside the tumor region to aid quantifying the immune-tumor cells colocalized at the tumor boundary. In the zoomed in region in the far left of FIG. 4A, three convex hull boundaries are shown using dotted lines. In some embodiments, regions 402 (i.e., tumor regions) are approximated using multiple convex hulls to allow for spatial quantification of tumor and immune cells within and outside the tumor border. For both pre-treatment and on-treatment patients, it has been found that there is a higher presence of T cells within the tumor and a higher abundance of tumor and immune cells across the tumor border in PD patients than in SD patients, signaling that there are distinct cellular compositions for PD and SD patients. This clustering is described in greater detail below with respect to FIGS. 6A-6C.

Referring now to FIG. 4B, box plots indicating the presence of Stromal T cells and T cells within tumors in both stable disease (SD) and progressive disease (PD) patients are shown, according to some embodiments. Specifically, the leftmost box plot shows Stromal T cells and T cells within tumors for pre-treatment patients whereas the rightmost box plot shows Stromal T cells and T cells within tumors for on-treatment patients. In both cases, it is shown that there is a higher presence of T cells in tumor in PD patients than SD patients. The Bonferroni-adjusted p-value with significance are: *: 1.00e-02<p<=5.00e-02, **:1.00e-03<p<=1.00e-02, ***:1.00e-04<p<=1.00e-03, ****: p<=1.00e-04.

Referring now to FIG. 4C, another box plot indicating the number of immune and tumor cells in a tumor border in both SD and PD patients is shown, according to some embodiments. In both pre- and on-treatment patients, there is a higher presence (with Bonferroni-adjusted p-values with significance) of PD-1, FoxP3, PanCK in PD patients than SD patients.

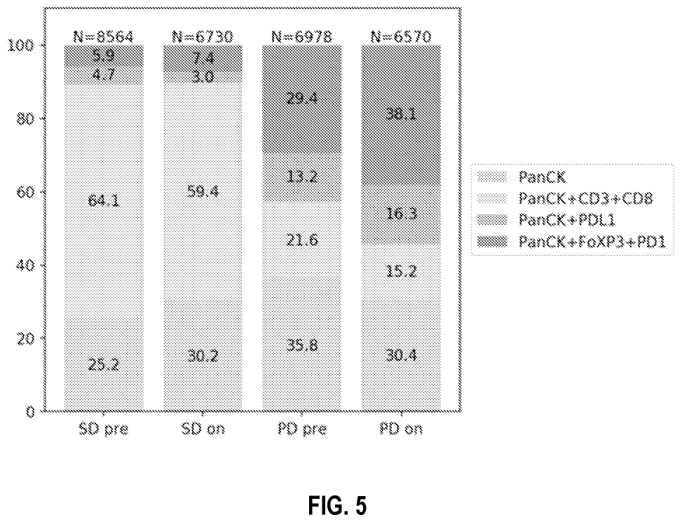

Referring now to FIG. 5, a graph indicating the relatively proportions of tumor cells colocalized with one of more functional markers with respect to patient response category and treatment timing is shown, according to some embodiments. Specifically, FIG. 5 shows the relative proportions of tumor cells (PanCK+) and tumor cells colocalized with one or more of the functional markers (CD3+ T cells, CD8+ T cells, FoXP3+(Treg cells), PD-L1, and PD-1) within each patient response category based on treatment timing. Pooled data from a plurality of patients are shown with cell numbers per category shown above the respective bar. FIG. 5 clearly illustrates the differences of tumor-immune cells across SD and PD patients.

To quantify the tumor-immune cell colocalization at the tumor border, single-cell analyzer 114 clusters the tumor-immune cell counts at the tumor border approximated using convex hulls, as described above with respect to FIG. 4A. Referring now to FIGS. 6A and 6B, t-distributed stochastic neighbor embedding (t-SNE) plots of three different clusters annotated with differentially-expressed markers are shown, according to some embodiments. Specifically, FIG. 6A shows a two-dimensional (2D) t-SNE plot of three different clusters of markers which are determined from the tumor-immune cell counts at the tumor border approximated by convex hulls, as discussed above with respect to FIG. 4A, where each point of the t-SNE of FIG. 6A abstracts an image. In some embodiments, clustering is performed using a Gaussian mixture model. It is shown that distinct clusters indicating higher colocalization of PanCK+PD-1+FoxP3 are present in PD patients and higher colocalization of PanCK+PD-L1 along with CD3+CD8+ immune cells are present in SD patients, further indicating underlying structural differences between PD and SD patient groups. FIG. 6B shows a disease response spread based on the t-SNE of FIG. 6A.

Referring now to FIG. 6C, an example t-SNE plot generated by replacing the points of the t-SNE plots of FIGS. 6A and 6B with corresponding multiplexed images is shown, according to some embodiments. This arrangement of images as an “image t-SNE” clearly highlights the difference in immune cell abundance across PD and SD patient groups. Specifically, FIG. 6C captures the immune cell gradient showing images higher in FoxP3+PD-1 colocalization to be generally observed across PD patients and images higher in CD3+CD8 colocalization to be generally observed across SD patients. FIG. 6C also helps in identifying groups of immunologically hot, warm, or cold tumors across patients.

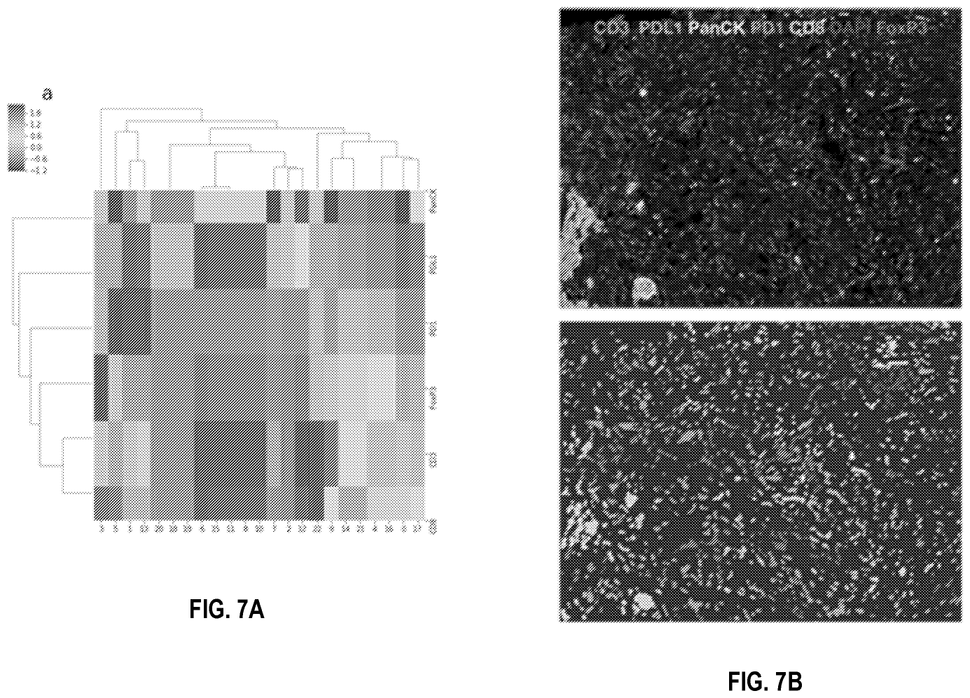

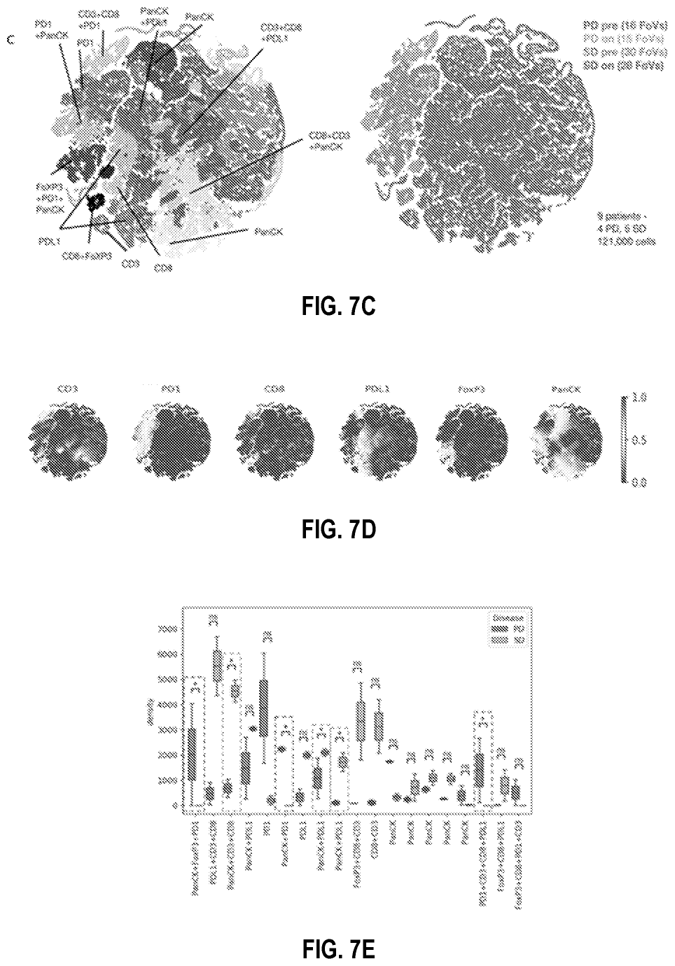

To further understand the spatial organization of tumor and immune cells across PD and SD patients, cell can be analyzed in the context of its spatial neighbors. This is achieved by generating (e.g., by single-cell analyzer 114) cellular neighborhoods, where the marker expression of each cell is the average of ten of its nearest spatial neighbors in Euclidean space. A cellular neighborhood can be defined as the minimal set of cell types that are both functionally and spatially similar. Referring now to FIG. 7A, a cluster gram illustrating multiple distinction cellular neighborhoods (CNs) compared to their z-scored frequencies in each CN is shown, according to some embodiments. Specifically, FIG. 7A shows 23 distinct cellular neighborhoods for an example image (x-axis) based on six distinct markers (y-axis), and illustrates the respective z-scored frequencies within each CN. Corresponding, FIG. 7B shows an example multiplexed field-of-view (FOV) with mapped CNs, according to some embodiments.

Referring now to FIG. 7C, a 2D t-SNE plot rendering of various cells colored by CN and patient category is shown, according to some embodiments. Specifically, the leftmost t-SNE of FIG. 7C shows approximately 121,000 cells separated by CN, whereas the rightmost t-SNE shows these cells separated by patient categories (e.g., SD on-treatment, SD pre-treatment, PD on-treatment, PD pre-treatment). In some embodiments, the different CNs or cells separated by patient categories are visually distinct, represented in FIG. 7C by different colors, shading, contrast, etc. Thus, from FIG. 7C, it is evident that there are characteristic cellular neighborhoods defining PD and SD patients such as PanCK+FoxP3+PD-1 for PD patients and PanCK+CD3+CD8 for SD patients. Those of skill in the art will appreciate that it has been previously shown that infiltration of intra-tumoral CD3+ and CD8+ T cells in NSCLC is associated with better survival outcome. It has also long been suggested that FoxP3 is a barrier to the efficacy of ICIs and therefore a potential target for therapy. Further, studies of NSCLC patients have shown that the spatial proximity of tumor and T regulatory cell predicts worse lung cancer survival.

Referring now to FIG. 7D, multiple versions of the t-SNE of FIG. 7C that each depict an expression spread of a different marker are shown, according to some embodiments. It should be noted that FIG. 7D includes the same t-SNE as FIG. 7C reproduced numerous times to depict the expression spread of six different markers. Referring now to FIG. 7E, a box plot of the density of different CNs based on patient category is shown, according to some embodiments. Specifically, FIG. 7E quantifies the frequencies (in terms of number of cells) of CNs in PD and SD patients showing statistically significant CNs (Bonferroni corrected, p-value<0.001).

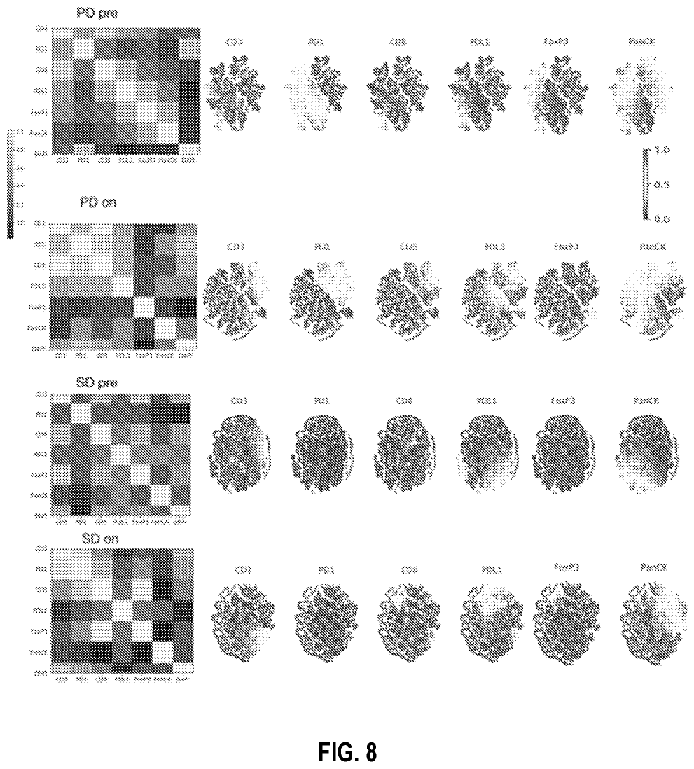

Referring now to FIG. 8, various plots of marker co-expression covariance captured by CNs across patient categories (e.g., SD on-treatment, SD pre-treatment, PD on-treatment, PD pre-treatment) and corresponding t-SNE plots of marker expressions based on patient category and treatment timing are shown, according to some embodiments. For marker co-expression covariances, each block of the plots identifies cells that both reside in spatially-similar neighborhoods while expressing similar markers. Lighter blocks indicate a high correlation (e.g., that cells are highly similar both spatially and functionally). In the corresponding marker t-SNE plots, high expression is indicated by lighter shading while low expression is indicated by darker shading.

### Predicting Disease Progression

As discussed above, the core function of single-cell analyzer 114, and more broadly system 100, is to predict whether a patient's NSCLC will progress or remain stable during treatment. To generate predictions, single-cell analyzer 114 includes a predictive model, such as an SVM classifier, which classifies image data into either a “progression” class or a “non-progression/stable” class. In some embodiments, the predictive model is trained using image data from the pre-treatment cases (e.g., for both SD and PD patients). The predictive model initially maps each data point of an image into a six-dimensional (6D) feature space (e.g., six being the number of markers used) and subsequently identifies the hyperplane that separates the data into the two classes while maximizing the marginal distance for both classes and minimizing the classification error. The marginal distance for a class is the distance between the decision hyperplane and its nearest instance which is a member of that class. Due to this formulation, an SVM classifier may be more accurate than other types of predictive models; however, it should be appreciated that other types of classifiers may be implemented by single-cell analyzer 114.

In one example, single-cell analyzer 114 may include 64 dense, 2D layers with a Rectified Linear Unit (ReLU) as the activation function. In some embodiments, the final 2D dense layer has a sigmoid activation with a L2 kernel regularizer. The model is trained for 20 epochs using a categorical hinge loss function and optimized using a stochastic gradient descent optimizer (e.g., Adadetla). In testing, the predictive model of single-cell analyzer 114 was trained on 47,000 pre-treatment cells, with the cells being randomly split into training, validation, and test sets. The predictive model is trained on the training set and predictions of disease progression using the test set. The response of the patients was known a priori.

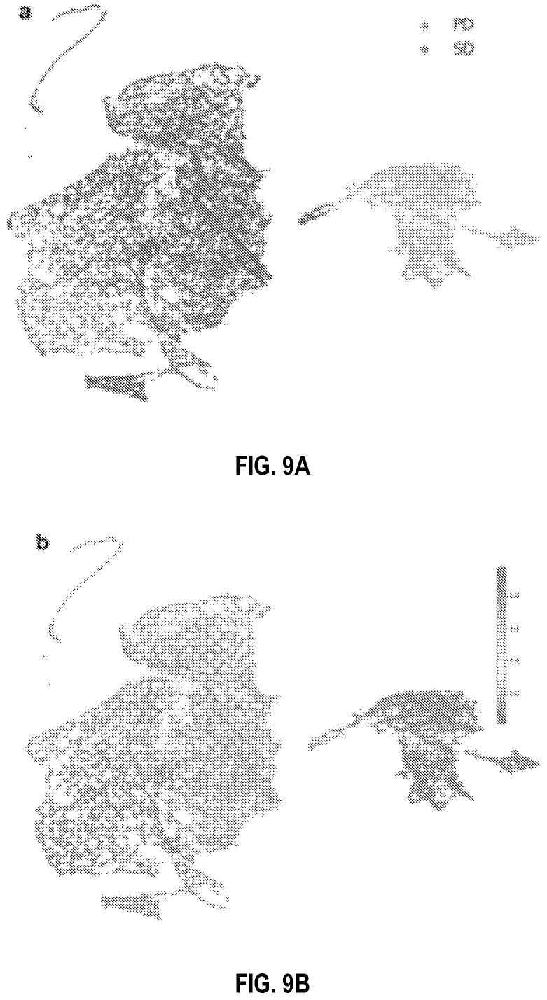

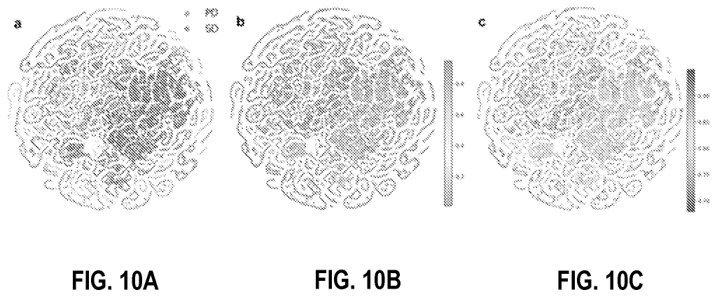

Referring now to FIGS. 9A and 9B, t-SNE plots of actual patient disease progression and predicted patient progression are shown, according to some embodiments. Specifically, FIG. 9A depicts a t-SNE projection of actual patient response based on pre-treatment data and FIG. 9B depicts the response predictions from single-cell analyzer 114 using the predictive model described herein. In some embodiments, the image of FIG. 9B is an example graphic that can be provided to a user via a user interface. In the example shown, the predictions are averaged across 10 independent runs by randomly shuffling all the test data points for each run. Classification accuracy is the fraction of correct predictions per run. Overall, FIGS. 9A and 9B show the single-cell analyzer 114 is able to predict disease progression with an accuracy of 94.25%, with class-specific accuracy of 95.4% for SD patients and 93.1% for PD patients. Referring also to FIGS. 10A-10C, t-SNE plots of actual patient disease progression, predicted patient progression, and single-cell model accuracy are shown for a second data set, according to some embodiments. In this example, disease progression was predicted for 98,000 pre-treatment cells.

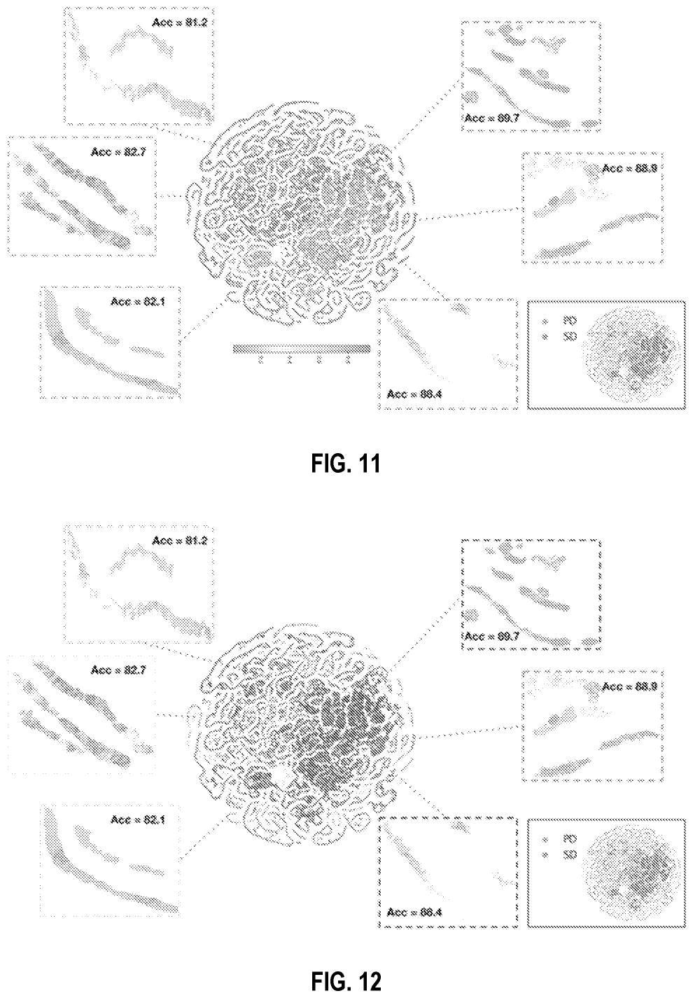

Referring now to FIGS. 11 and 12, risk maps generated based on predicted disease progression are shown, according to some embodiments. Risk maps show the probability of disease progression for a given patient or group of patients along with the patient's overall chance to progress, and are oriented based on the patient response category. The probabilities of progression are well aligned to each patient's response where patient response is shown in the inset. PD patents are predicted to show higher progression, denoted by darker shades and SD patients show lower progression, denoted by lighter shades. Prediction accuracy for each patient is given within the risk map. Each risk map is outlined based on the patient response category.

### Disease Progression Prediction

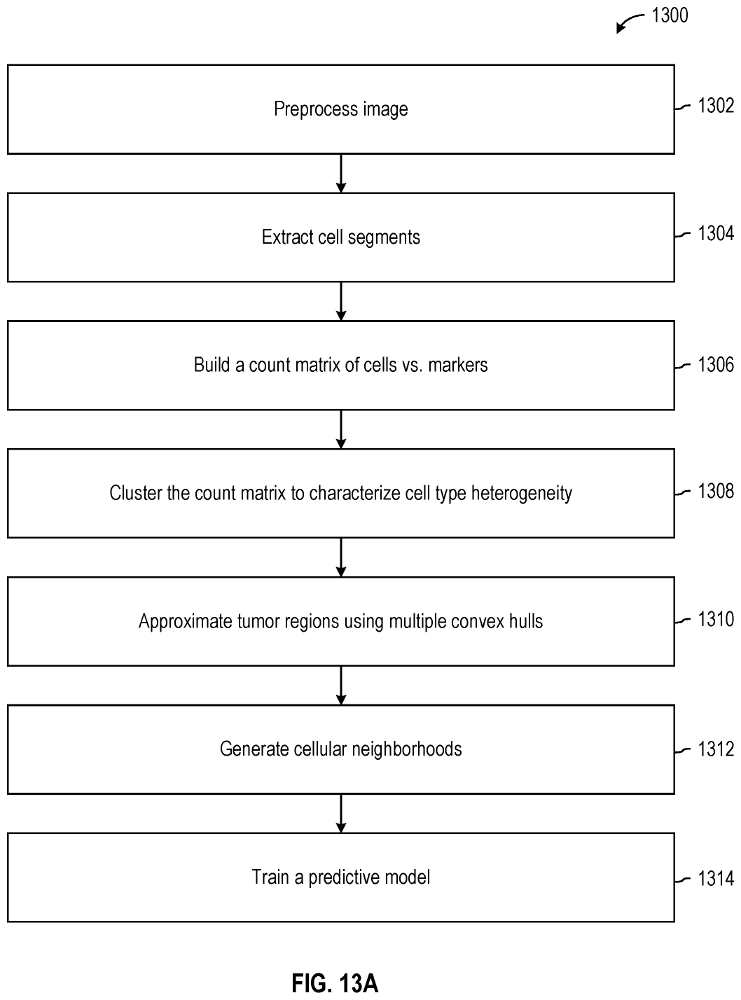

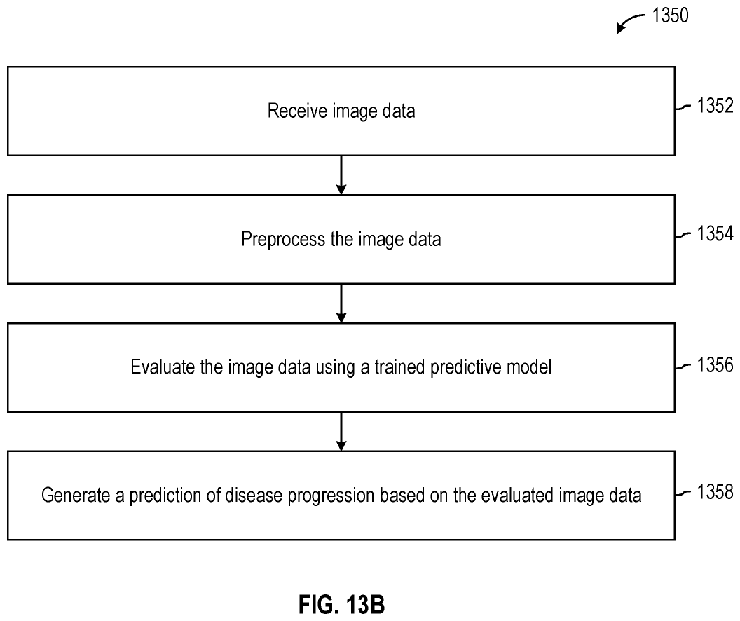

Referring now to FIG. 13A, a flow chart of a process 1300 for processing image data and training a predictive model is shown, according to some embodiments. Specifically, process 1300 may be implemented to train the predictive model of single-cell analyzer 114, as described above. Accordingly, in some embodiments, process 1300 is implemented by system 100 and, more specifically, single-cell analyzer 114. It will be appreciated that certain steps of process 1300 may be optional and, in some embodiments, process 1300 may be implemented using less than all of the steps.

At step 1302, an image or a set of images is received and preprocessed. As described above, the image(s) are typically multiplexed images stained for various markers, including one or more of CD3, PDL1, Pan-CK, PD1, CD8, DAPI, and FoxP3. In some embodiments, image data is received (or retrieved from a database) in a TIFF format, although other formats may be used. In some embodiments, a multiplexed image includes multiple individual image files (e.g., multiple TIFFs). Each file may be associated with one type of marker, as mentioned above. In some embodiments, includes combining (i.e., aggregating) multiple images to form a multiplexed image. In some embodiments, preprocessing includes denoising (i.e., cleaning) each image. For example, Otsu's method of automatic image thresholding, which involves iterating through all possible threshold values and calculating a measure of spread for the pixel levels each side of the threshold (e.g., the pixels that either fall in foreground or background), is used to clean image data. Foreground pixels may be regarded as true stain signals while background pixels may be interpreted as noise. Thus, background pixels can be filtered out or ignored. In some embodiments, preprocessing also includes generating a grayscale version of each image. In some embodiments, preprocessing includes tiling each image into smaller frames (e.g., 256×256 pixel frames).

At step 1304, cell segments are extracted from the multiplexed and (optionally) preprocessed image data. In some embodiments, the image(s) are fed into a deep-learning architecture for segmentation. Specifically, the image(s) may be fed into a convolutional neural network, such as U-Net, which predicts the cell segments (e.g., the nuclei of the cells) in the images. The identified cell segments can then be stitched together.

At step 1036, a count matrix of cells versus markers is built. The count matrix may indicate cells as rows and markers as columns. Subsequently, at step 1308, the count matrix is clustered to characterize cell type heterogeneity (e.g., to identify heterogeneous cell types). In some embodiments, cluster assignments are determined using a Gaussian mixture model which identifies heterogeneous cell types based on stain expression and, advantageously, does not require the number of clusters to be known in advance.

Based on the determined heterogeneous cell types (e.g., from clustering), at step 1310, tumor regions are approximated using multiple convex hulls. In some embodiments, tumor-rich regions (e.g., which are higher in PanCK expression) are automatically demarcated, as shown in FIG. 4A above. At step 1312, cellular neighborhoods are generated or identified. A cellular neighborhood can be defined as the minimal set of cell types that are both functionally and spatially similar. Finally, at step 1314, the predictive model is trained using the determined cellular neighborhoods. More generally, the predictive model is trained using historical data (e.g., previously-captured images) to determine whether a patient's NSCLC will progress during treatment.

Referring now to FIG. 13B, a flow chart of a process 1350 for predicting disease progression using a cell-segmentation approach is shown, according to some embodiments. In some embodiments, process 1350 is implemented after training a predictive model (e.g., the SVM classifier of single-cell analyzer 114) using process 1300. In some embodiments, process 1350 is implemented by system 100, as described above. It will be appreciated that certain steps of process 1350 may be optional and, in some embodiments, process 1350 may be implemented using less than all of the steps.

At step 1352, image data is received. In some embodiments, image data is received from a medical imaging device. In some embodiments, image data is stored in a database and retrieved at step 1352. Subsequently, at step 1354, the image data is preprocessed using any of the preprocessing techniques described above with respect to step 1302 of process 1300. For the sake of brevity, the preprocessing techniques described above are not repeated here.

At step 1356, the image data is evaluated using a trained predictive model. In some embodiments, the predictive model is trained by process 1300. As discussed above, the predictive model may be a classifier, such as an SVM classifier, that classifies image data into one of a “progression” or a “non-progression/stable” class. In some embodiments, the predictive model classifies an entire image as belonging to a patient with progressive NSCLC or a patient with stable NSCLC. In other embodiments, the predictive model identifies individual cells in the image that are predicted to progress throughout treatment. To this point, at step 1358, disease progression is predicted based on the evaluated image data. In some embodiments, step 1358 also includes providing a report or alert to a user of system 100 (e.g., a medical professional). For example, system 100 may display a user interface that indicates whether disease progression is predicted. In another example, system 100 may transmit an email, text message, push notification, or the like to a user's personal electronic device (e.g., cell phone, smartwatch, personal computer, etc.) informing the user to the prediction.

In some embodiments, process 1350 further includes a step of administering treatment to the patient based on the prediction of whether the patient's NSCLC will progress. In some such embodiments, administering treatment can includes starting, stopping, or altering an NSCLC treatment regimen. For example, the dosage of one or more medications may be adjusted or the prediction may inform a medical professional that a particular medication may not be effective, such that an alternative treatment or medication is selected.

### Quadrant Analysis

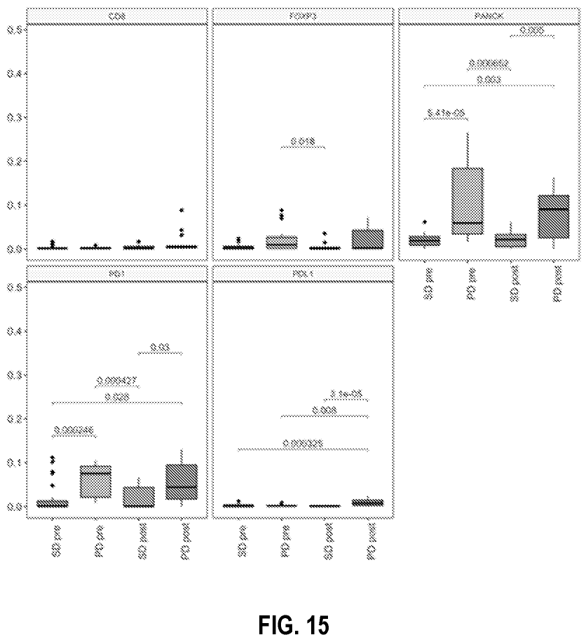

In a parallel and complementary approach to the cell-segmentation method described above, quadrant analyzer 116 implements a species distribution model (SDM) that provides a framework to infer and explain species distribution and detect and predict global changes in species ecologies. The fundamental modeling steps in SDMs are the data preparation, model fitting and assessment, and prediction. As described above with respect to FIGS. 1 and 2, using the quadrant approach, each patient sample (i.e., each image) is converted to a collection of fixed-size grids, called quadrants, rather than as a collection of single cells. Thus, each quadrant can be examined to identify ecology changes between PD and SD patients. In particular, each quadrat expresses the area normalized sum of marker intensity values of all cells present within that quadrant. The quadrants of an image are combined and fit using a Gaussian copula graphical lasso (GCGL) for both the pre- and on-treatment groups, as shown in FIG. 14A described below. Since the copula can directly model covariance and strength between markers, the GCGL creates a single association network with all direct links between markers and strength of association increases from red to blue. It has been observed that, during the course of treatment, the spatial organization has changed in PD with FoxP3 becoming strongly associated with tumor, as shown in FIG. 15, also described below.

Next, to predict the probability of disease progression, quadrant analyzer 116 implements a boosted regression tree (BRT). The BRT can be trained using quadrat counts (e.g., 100 um×100 um) from patients in the pre-treatment category. BRTs are augmented regression trees that can associate a response variable (e.g., disease progression) with predictor variables (e.g., marker expressions) by recursively splitting and combining parsimonious trees to generate disease progression predictions. Using these quadrat intensities, quadrant analyzer 116 predicts whether or not a given quadrat came from a patient that progressed while on treatment. Subsequently a risk map, similar to those shown in FIGS. 11 and 12, that identifies areas of the tumor associated with high probabilities of progression while on treatment can be generated.

Referring now to FIG. 14A, a diagram of the spatial association between markers across patient categories is shown, according to some embodiments. Specifically, FIG. 14A shows networks of marker associations obtained using the GCGM mentioned above, shown per patient category. FIG. 14B shows progression probability predictions obtained by a BRT using pre-treatment marker co-localizations in quadrants, as discussed briefly above. FIG. 14C show multiple graphs of marker importance, according to some embodiments. The leftmost graph quantified marker importance based on the number of times it is selected for splitting, weighted by the squared improvement to the model as a result of each split, and averaged over all trees by quadrant analyzer 116. The middle graph indicate the number of markers in each quadrat plotted against probability of progression. The rightmost graph indicates marker co-interaction across pre- and on-treatment cases. Finally, FIG. 14D shows graphs of progression probability scores mapped onto quadrants of example SD and PD images, according to some embodiments.

Referring now to FIG. 15, a graph of marker abundance across all FOV quadrants is shown, according to some embodiments. Specifically, FIG. 15 shows marker abundance per patient response category. As shown, PD-1, FoxP3, and PanCK have a higher presence in PD patients than in SD patients.

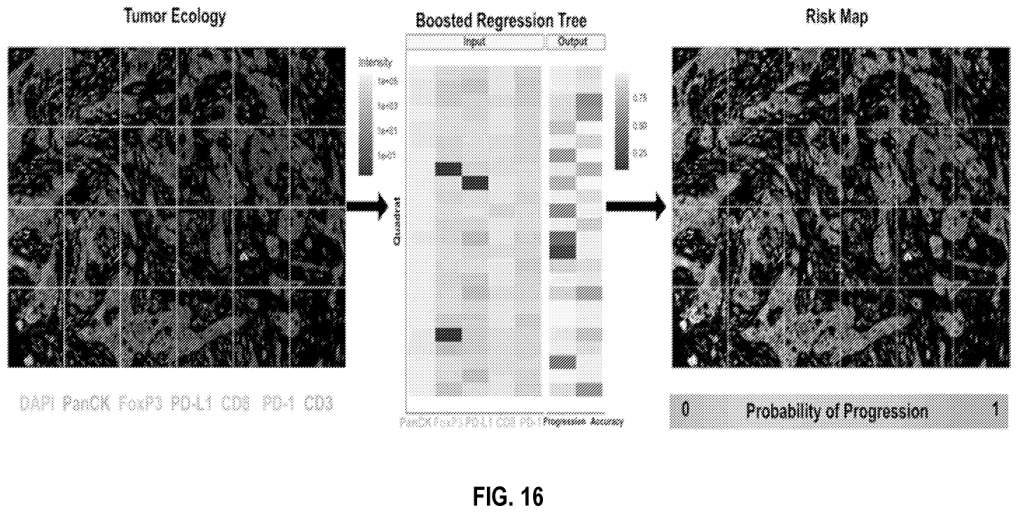

Referring now to FIG. 16, a risk map for an example PD patient is shown, according to some embodiments. More generally, FIG. 16 shows a risk map generated by feeding an example through a BRT (e.g., of quadrant analyzer 116). As shown, the spatial co-localization of cells within each 100 μm×100 μm quadrat from pre-treatment biopsies (left) can be used to train a BRT (middle) that predicts the probability a patient will progress while on treatment. Quadrant analyzer 116 can then project the probabilities back on to the image, potentially identifying regions of interest in a risk map (right). The example images shown are from one FOV from a PD patient.

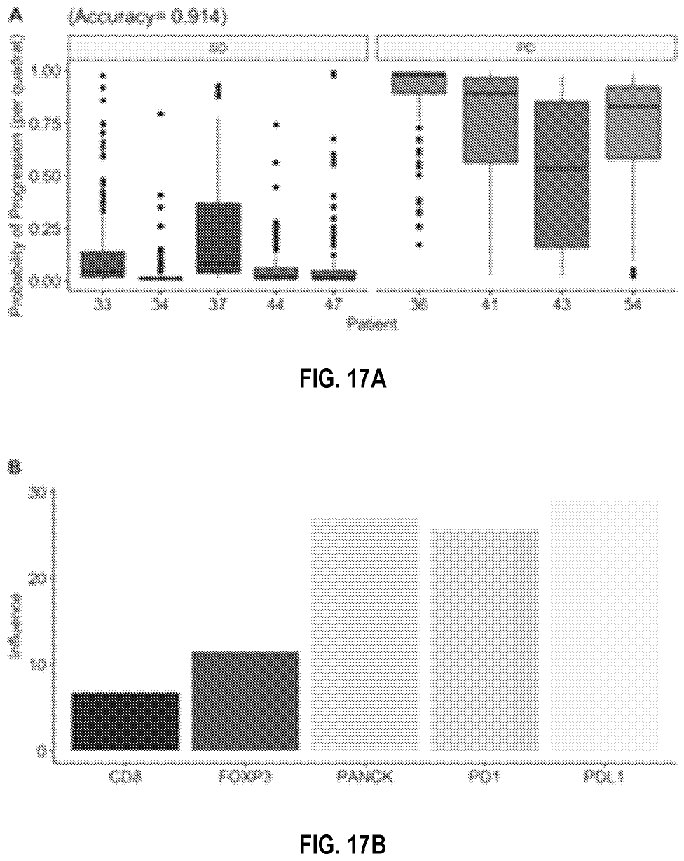

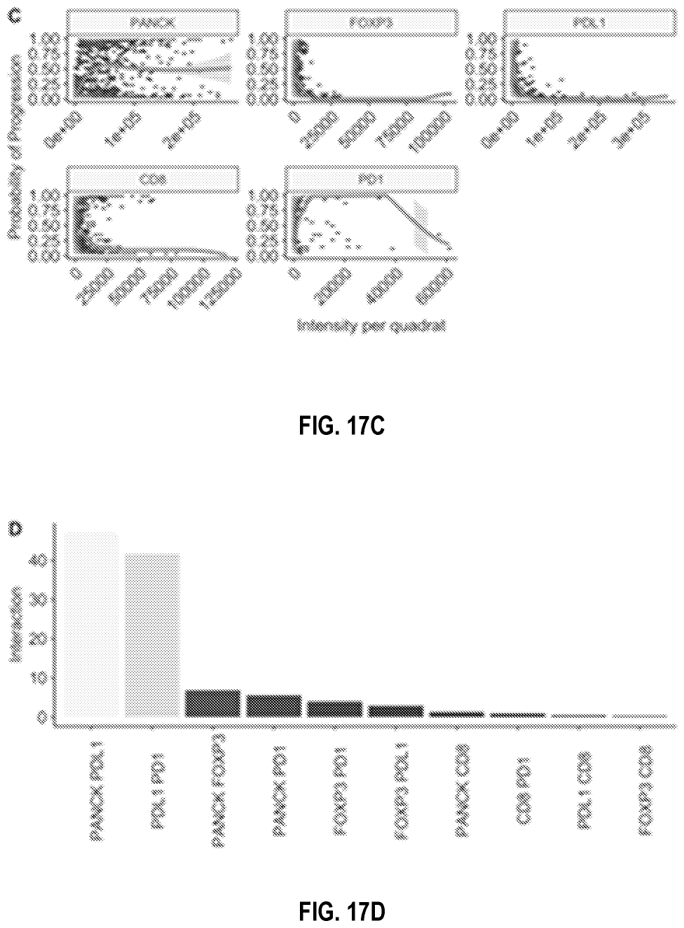

Referring now to FIG. 17A, a graph of the probability of disease progression per quadrant is shown for an example image, according to some embodiments. In some embodiments, the probabilities graphed in FIG. 17A represent the probability of disease progression as predicted by quadrant analyzer 116. As shown, quadrant analyzer 116 was able to correctly predict whether or not a quadrant of an image came from a patient with PD 91.4% of the time. The distribution of quadrat probabilities could be used to assess risk of progression, as on average patients with SD had lower quadrat probabilities than PD patients. In addition to making spatial predictions, the distribution of quadrant probabilities can be used to make an overall prediction of how the patient will respond to therapy. It is evident that by combining quadrants for each of the SD and PD groups, the regression trees with k-fold cross validation are able to predict disease progression with a high accuracy. These results suggest that spatial measurements of the tumor ecology, and thus the interactions between cell types, can be used to make accurate predictions of how a patient will respond to therapy.

Referring now to FIG. 17B, a graph of feature importance in predicting disease progression using the quadrant approach is shown, according to some embodiments. Specifically, FIG. 17B illustrates a feature importance analysis which reveals that PD-L1, PD-1, and PanCK had the greatest impact on the ability of quadrant analyzer 116 to accurately predict treatment response, suggesting these markers play the most important roles in determining whether or not a patient responds to treatment. In addition to prediction response, it was found that PD-L1 is the most important variable in predicting response, closely followed by PanCK (tumor) and PD-1.

Referring now to FIG. 17C, variable response plots that indicate the risk of disease progression based on different markers are shown, according to some embodiments. As shown, risk of progression increases with higher levels of tumor (PanCK) and PD-1 but decreases as levels of PD-L1 increase. By examining the response of each variable (i.e., marker) as shown in FIG. 17, one can see that high levels of PD-1 and PanCK are associated with higher probabilities of progression, while high levels of PD-L1 are associated with lower probabilities of progression.

Finally, it is also possible to examine the interactions of the markers, based on their co-localization within each quadrat, as shown in FIG. 17D which includes a graph illustrating the interaction effects between variable markers, according to some embodiments. In FIG. 17D, it is shown that the mix of PanCK and PD-L1, and the mix of PD-L1 and PD-1, have the strongest impact on predictions, suggesting that these pairs of markers strongly influence whether or not a patient responds to therapy. By collectively examining each marker's importance, response, and interactions, a picture emerges wherein a distinguishing factor between PD and SD is that the tumors of the former are characterized by a more highly suppressed immune response prior to treatment. This in turn may help explain why these patients have a higher probability of progressing while on treatment.

### Predicting Disease Progression

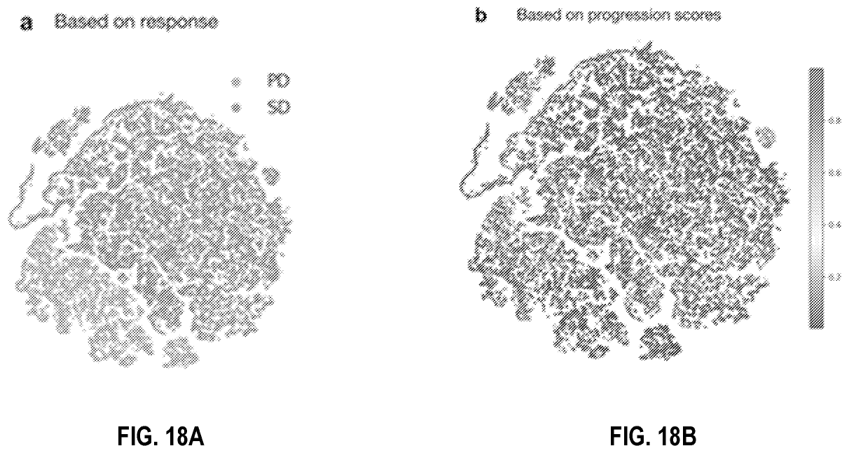

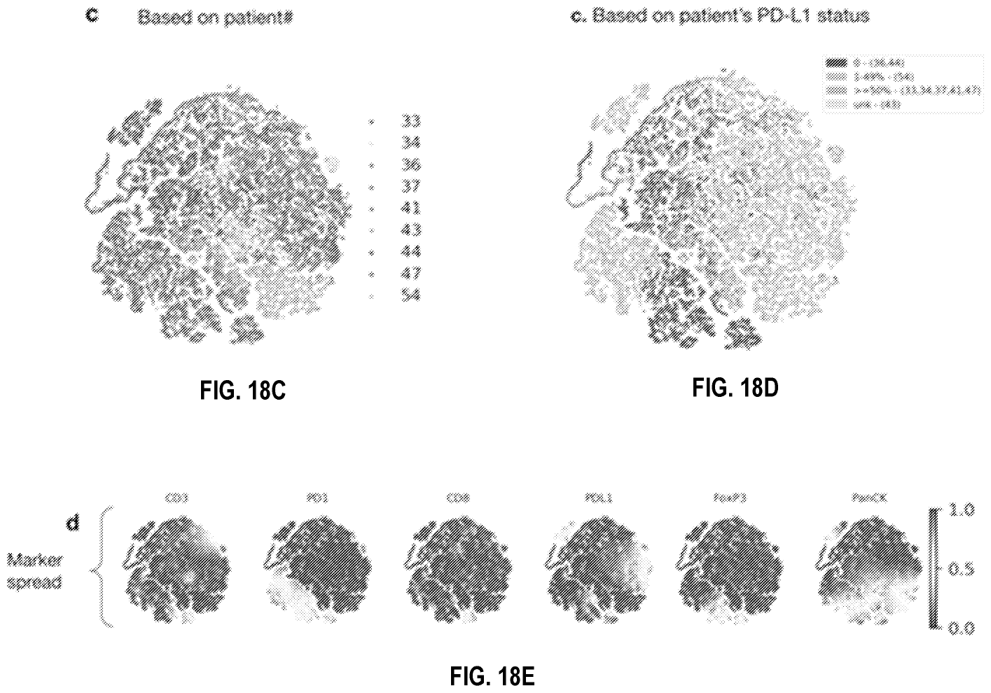

Referring now to FIGS. 18A-18D, t-SNE plots illustrating actual patient response and predicted progression scores are shown, according to some embodiments. In some embodiments, quadrants are defined at two different levels of granularity: a finer granularity where each quadrat is a cell with its neighborhoods and a coarser granularity where each quadrat is a fixed-size grid with a collection of cells and their neighborhoods. In the finer granularity case, by clustering cells and projecting the cells onto a 2D t-SNE, a clear distinction between PD and SD regions is shown, as depicted in FIG. 18A.

Next, to depict disease prediction, a BRT is trained (e.g., by quadrant analyzer 116) and tested in a cross-validation setting. For this, cells are randomly split into training, validation, and test sets, and the training set is used as input to the predictive model of quadrant analyzer 116. The predictive model may then output predictions of disease progression based on the test set. This process of training and generating predictions is iterated, ensuring that all cells have partaken at least once in the validation and test sets. The disease progression prediction scores obtained from the predictive model can be averaged using Lowes smoothing, as shown in FIG. 18B. As shown, the prediction score overlap with the PD and SD regions in FIG. 18A, showing high progression probability corresponding to the PD region.

Referring now to FIG. 18E, various plots of marker co-expression covariance captured by CNs across patient categories and corresponding t-SNE plots of marker expressions based on patient category and treatment timing are shown, according to some embodiments. In the marker spread of FIG. 18E, the marker triplet (PD-1+PanCK+FoxP3) is differentially expressed in the PD region, indicating a more highly suppressed immune environment, aiding disease progression.

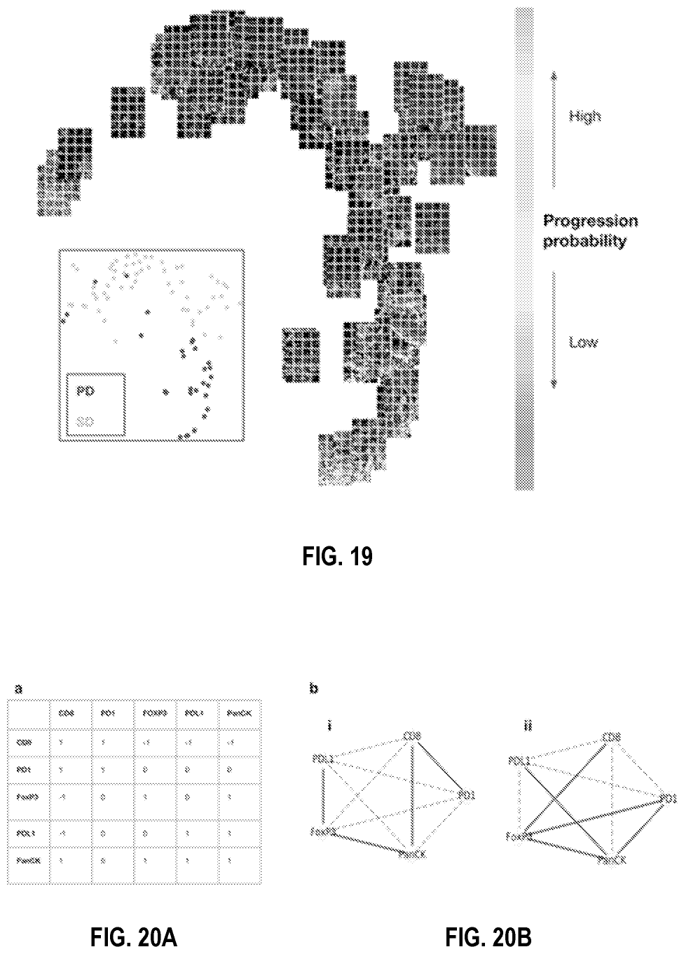

Referring now to FIG. 19, an example t-SNE plot generated by replacing the points with corresponding multiplexed images is shown, according to some embodiments. Specifically, each image of FIG. 19 is colored or shaded, per quadrat, using the probability of progression scores calculated by quadrant analyzer 116. In this example, lighter shading indicates a higher likelihood of disease progression while darker shading denotes a lower chance of progression.

In order to quantify the distinct architectures across PD and SD patients, a statistical model can be built to infer the difference in marker interactions, in the form of a network, between patient categories. This is done by utilizing the marker expression both at the pre-treatment and during-treatment states for each category and capturing the “difference network” that potentially drives the patients from the pre-treatment state to the on-treatment state. The inference problem can be stated as a least-squares minimization problem and can be further regularized using prior biological knowledge of marker interactions.

We denote the change in the i^{th} marker expression in the pre-treatment case at time t, mᵢ^{pre}(t) as:

$$\frac{d {m}_{i}^{p r e} ( t )}{d t} = {\sum }_{j = 1}^{n} {W}_{ij} {m}_{j}^{p r e} ( t ) - {m}_{i}^{o n} ( t )$$

where n is the number of markers, i, j=1, 2, . . . , n, and W is the weight matrix. Equation 1 can be written in matrix form as:

$$\frac{{dM}^{p r e} ( t )}{dt} = {WM}^{p r e} ( t ) - {M}^{o n} ( t )$$

where M*(t)=m₁*(t), m₂*(t), . . . , mₙ*(t) and * denotes pre or on states. Equation 2 can be solved as a least-squares minimization problem given n markers and (Mᵢ^{pre}, Mᵢ^{on}) as steady states. This can be written as:

$${W}^{L S} = arg {min}_{W} f ( W ) = arg {min}_{W} {\sum }_{j = 1}^{n} ‖ {WM}^{p r e} - {M}^{o n} ‖$$

Further, prior knowledge of biological mechanisms between markers can be added as additional constraints to the least-squares problem. These mechanisms can be captured in pairwise format as a matrix W^{prior}, as shown in FIG. 20A, where a ‘1’ indicates positive interaction, a ‘0’ indicates no interaction, and a ‘−1’ indicates negative interaction. Equation 3 can be written as follows to yield W^{LSprior}:

$${w}^{LSprior} = arg {min}_{W} f ( w ) = arg {min}_{W} {\sum }_{j = 1}^{n} ‖ {WM}^{p r e} - {M}^{o n} ‖ + λ ‖ W ∘ {W}^{p r i o r} ‖$$

Network depictions of marker associations are also shown in FIG. 20B, according to some embodiments, where W^{LSprior} is depicted as a network for SD (i) and PD (ii) obtained by solving Equation 4, above. W^{LSprior} maps the markers from the “pre” to the “on” state while incorporating known mechanisms as prior information. Solid lines indicate positive marker associations and dashed lines indicate negative associations.

In some embodiments, count matrices can be constructed for the four categories: SD (pre- and on-treatment) and PD (pre- and on-treatment) based on cell segments. Assuming that each marker follows a Gaussian distribution, count matrices of cells x markers can be modeled to follow a multivariate Gaussian distribution without loss of generality. In some embodiments, first and second order moments are computed to describe each of the four categories. Next, for each category, 10,000 samples are randomly select to create a random matrix of 10,000 rows and five markers. This can be repeated 100 times and the random matrices averaged out to create one representative matrix denoting that category. This is done to ensure that the random matrices that have the same number of rows in the ‘pre’ and ‘on’ states and that obey the observed/empirical distributional moments. Implementing Equation 4, above, by incorporating the biological prior yield W^{LSprior} for SD and PD (e.g., FIG. 20B, i and ii) respectively. It becomes evident through WW^{LSprior} for SD that there is increased CD8 infiltration into the tumor with decreased PD-L1 expression.

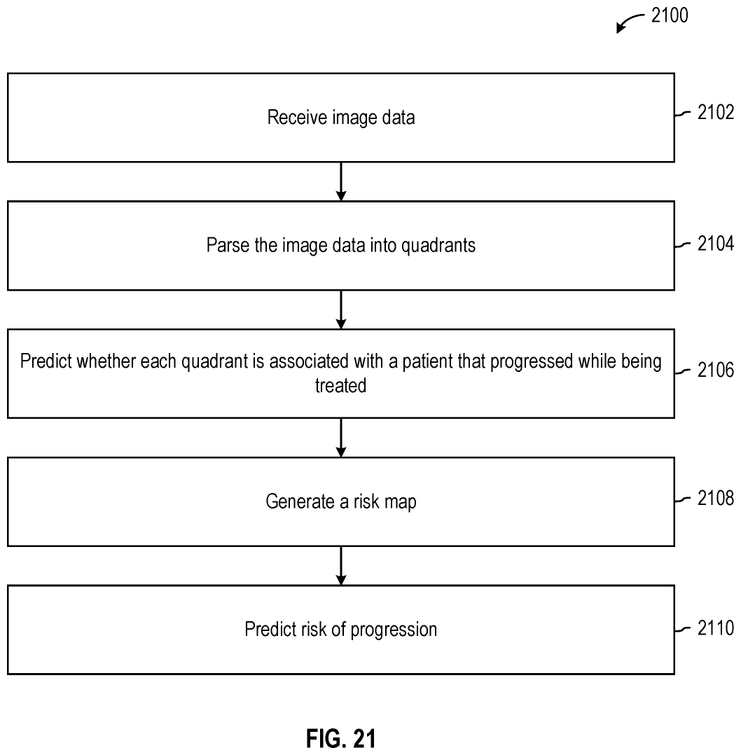

Referring now to FIG. 21, a flow chart of a process 2100 for predicting disease progression using a quadrant approach is shown, according to some embodiments. In some embodiments, process 2100 is implemented by system 100, as described above. More specifically, process 2100 may be implemented by quadrant analyzer 116. In some embodiments, process 2100 is performed concurrently with, or before or after, processes 1300 and 1350, described above. For example, process 1350 and process 2100 may be considered complementary processes that are executed simultaneously or in succession. It will be appreciated that certain steps of process 2100 may be optional and, in some embodiments, process 2100 may be implemented using less than all of the steps.

At step 2102, image data is received. In some embodiments, image data is received from a medical imaging device. In some embodiments, image data is stored in a database and retrieved at step 2102. In some embodiments, the image data is also preprocessed after receiving using any of the preprocessing techniques described above with respect to step 1302 of process 1300. For the sake of brevity, the preprocessing techniques described above are not repeated here.

At step 2104, the image data is parse into quadrants. As described above, quadrants are equally-sized “tiles” that separate the image into several smaller images. Then, at step 2106, each quadrant is fed into a BRT to predict whether a probability of NSCLC progression in each quadrant. At step 2108, a risk map is generated based on the predicted disease progression for each quadrant. A risk map shows the probability of disease progression for the patient along with the patient's overall chance to progress. Finally, at step 2110, the risk of NSCLC is predicted. In some embodiments, step 2110 also includes providing a report or alert to a user of system 100 (e.g., a medical professional). For example, system 100 may display a user interface that indicates whether disease progression is predicted. In another example, system 100 may transmit an email, text message, push notification, or the like to a user's personal electronic device (e.g., cell phone, smartwatch, personal computer, etc.) informing the user to the prediction.

In some embodiments, process 2100 further includes a step of administering treatment to the patient based on the prediction of whether the patient's NSCLC will progress. In some such embodiments, administering treatment can includes starting, stopping, or altering an NSCLC treatment regimen. For example, the dosage of one or more medications may be adjusted or the prediction may inform a medical professional that a particular medication may not be effective, such that an alternative treatment or medication is selected.

### Example Embodiments Using Machine Learning Models to Process Medical Images

Example methods of processing medical image data to predict disease progression in non-small cell lung cancer (NSCLC) patients using machine learning models are now described. A method can include receiving a multiplexed tissue image comprising a plurality of cells stained for one or more markers. This process is described in detail above. Additionally, the method can include evaluating the multiplexed tissue image using a machine learning model. Further, the method can include predicting whether a patient's NSCLC will progress based on the evaluation of the multiplexed tissue image using the machine learning model.

The term “machine learning” is defined herein to be a subset of artificial intelligence that enables a machine to acquire knowledge by extracting patterns from raw data. Machine learning techniques include, but are not limited to, logistic regression, support vector machines (SVMs), decision trees, Naïve Bayes classifiers, and artificial neural networks. The term “deep learning” is defined herein to be a subset of machine learning that that enables a machine to automatically discover representations needed for feature detection, prediction, classification, etc. using layers of processing. Deep learning techniques include, but are not limited to, artificial neural network or multilayer perceptron (MLP).

Machine learning models include supervised, semi-supervised, and unsupervised learning models. In a supervised learning model, the model learns a function that maps an input (also known as feature or features) to an output (also known as target or target) during training with a labeled data set (or dataset). In an unsupervised learning model, the model learns a function that maps an input (also known as feature or features) to an output (also known as target or target) during training with an unlabeled data set. In a semi-supervised model, the model learns a function that maps an input (also known as feature or features) to an output (also known as target or target) during training with both labeled and unlabeled data.

In some implementations, the machine learning model is a supervised machine learning model. For example, the machine learning model may optionally be a classifier model, where the classifier model classifies each of the plurality of cells as either stable or progressive. An example classifier model is a support vector machine (SVM) classifier. Use of SVMs is described in detail above. It should be understood that an SVM is provided only as an example classifier model. This disclosure contemplates using other types of machine learning classifiers with the techniques described herein.

Alternatively, the machine learning model may optionally be a regressor model, wherein the regressor model outputs a probability of NSCLC progression. An example regressor model is a boosted regression tree (BRT). Use of BRTs are described in detail above. It should be understood that a BRT is provided only as an example regressor model. This disclosure contemplates using other types of machine learning regressors with the techniques described herein.

### Configuration of Exemplary Embodiments

The construction and arrangement of the systems and methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative embodiments. Other substitutions, modifications, changes, and omissions may be made in the design, operating conditions, and arrangement of the exemplary embodiments without departing from the scope of the present disclosure.

The present disclosure contemplates methods, systems, and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products including machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures, and which can be accessed by a general purpose or special purpose computer or other machine with a processor.

When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general-purpose computer, special purpose computer, or special purpose processing machines to perform a certain function or group of functions.

Although the figures show a specific order of method steps, the order of the steps may differ from what is depicted. Also, two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps and decision steps.

It is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes¬ from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

1. (canceled)

2. (canceled)

3. (canceled)

4. (canceled)

5. (canceled)

6. (canceled)

7. A method of processing medical image data to predict disease progression in non-small cell lung cancer (NSCLC) patients, the method comprising:
receiving a multiplexed tissue image comprising a plurality of cells stained for one or more markers;
evaluating the multiplexed tissue image using a machine learning classifier model, wherein the machine learning classifier model classifies each of the plurality of cells as either stable or progressive; and
predicting whether a patient's NSCLC will progress based on the evaluation of the multiplexed tissue image using the machine learning classifier model.

8. The method of claim 7, further comprising preprocessing the multiplexed tissue image prior to evaluating the multiplexed tissue image, wherein preprocessing comprises at least one of:
denoising the multiplexed tissue image using Otsu's method of automatic image thresholding;
converting the multiplexed tissue image to grayscale; and
tiling the multiplexed tissue image into a plurality of n pixel by m pixel frames, where n and m are integers greater than 0.

9. The method of claim 7, wherein evaluating the multiplexed tissue image further comprises, prior to classifying the plurality of cells:
extracting cell segments from the multiplexed tissue image using a convolutional neural network;
building a count matrix that compares the plurality of cells to the one or more markers from the extracted cell segments;
clustering the count matrix to characterize cell type heterogeneity using a Gaussian mixture model;
approximating tumor regions from the characterized cell types using multiple convex hulls; and
identifying cellular neighborhoods based on the tumor regions.

10. The method of claim 7, wherein the multiplexed tissue image is a 7-stain image.

11. The method of claim 7, wherein the multiplexed tissue image is received from one of a medical imaging device or a database.

12. The method of claim 7, further comprising presenting an indication of the prediction to a user via a user interface.

13. The method of claim 7, further comprising generating a risk map that indicates a probability of NSCLC progression based on the prediction.

14. The method of claim 7, wherein the machine learning classifier model is a support vector machine (SVM).

15. The method of claim 7, further comprising:
parsing the multiplexed tissue image into a plurality of quadrants; and
evaluating each of the plurality of quadrants using a boosted regression tree (BRT), wherein the prediction of whether the patient's NSCLC will progress is further based on an output of the BRT, and wherein the BRT outputs a probability of NSCLC progression for each of the plurality of quadrants.

16. The method of claim 7, further comprising:
administering treatment to the patient based on the prediction of whether the patient's NSCLC will progress.

17. The method of claim 16, wherein administering treatment comprises starting, stopping, or altering an NSCLC treatment regimen.

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. A system for processing medical image data related to non-small cell lung cancer (NSCLC), the system comprising:
at least one processor; and
memory having instructions stored thereon that, when executed by the at least one processor, cause the system to perform operations comprising:
receiving a multiplexed tissue image comprising a plurality of cells stained for one or more markers;
evaluating the multiplexed tissue image using one of a support vector machine (SVM) classifier or a boosted regression tree (BRT), wherein the SVM classifier classifies each of the plurality of cells as either stable or progressive, and wherein the BRT outputs a probability of NSCLC progression for each of a plurality of quadrants parsed from the multiplexed tissue image; and
predicting whether a patient's NSCLC will progress based on the evaluation of the multiplexed tissue image using the SVM classifier or the BRT.

30. The system of claim 29, wherein the operations further comprise preprocessing the multiplexed tissue image prior to evaluating the multiplexed tissue image, wherein preprocessing comprises at least one of:
denoising the multiplexed tissue image using Otsu's method of automatic image thresholding;
converting the multiplexed tissue image to grayscale; and
tiling the multiplexed tissue image into a plurality of n pixel by m pixel frames, where n and m are integers greater than 0.

31. The system of claim 30, wherein the operations further comprise presenting an indication of the prediction to a user via a user interface.

32. The system of claim 30, wherein the operations further comprise generating a risk map that indicates a probability of NSCLC progression based on the prediction.

33. The system of claim 30, wherein the multiplexed tissue image is a 7-stain image.

34. The system of claim 30, wherein the multiplexed tissue image is received from one of a medical imaging device or a database.

35. The system of claim 30, wherein the multiplexed tissue image comprises a plurality of individual image files each associated with a single biomarker.

36. The system of claim 30, wherein evaluating the multiplexed tissue image further comprises, prior to classifying the plurality of cells:
extracting cell segments from the multiplexed tissue image using a convolutional neural network;
building a count matrix that compares the plurality of cells to the one or more markers from the extracted cell segments;
clustering the count matrix to characterize cell type heterogeneity using a Gaussian mixture model;
approximating tumor regions from the characterized cell types using multiple convex hulls; and
identifying cellular neighborhoods based on the tumor regions.

37. The system of claim 36, wherein the operations further comprise:
training the SVM classifier or the BRT using the identified cellular neighborhoods.
